(19) United States
(12) Patent Application Publication
(10) Pub. No.: US 2022/0040424 A1
(43) Pub. Date: Feb. 10, 2022

(54) NASAL CAVITY SAMPLING METHODS AND APPARATUS

(71) Applicant: Rocket Science Health Corp., Vancouver (CA)

(72) Inventors: David James ALT, North Vancouver (CA); Kenneth Colin MacNarin IRVING, Victoria (CA); James Patrick JACKSON, Victoria (CA); Peter OXLEY, Rothesay (CA); Kenza Elizabeth COUBROUGH, Seattle, WA (US); Alexandra REID, Victoria (CA); Josh Adrian COUTTS, Victoria (CA); Aaron Olafur Laurence PHILIPPSEN, Victoria (CA); Nicholas David ALLAN, Victoria (CA); Meghan ROBINSON, Victoria (CA); Evan MCCORDICK, Rye, NY (US)

(21) Appl. No.: 17/503,090

(22) Filed: Oct. 15, 2021

Related U.S. Application Data
(63) Continuation of application No. PCT/IB2020/000293, filed on Apr. 23, 2020.
(60) Provisional application No. 62/838,169, filed on Apr. 24, 2019.

(51) Int. Cl. A61M 15/08; G01N 33/569; G01N 33/68

(52) U.S. Cl. CPC A61M 15/08; G01N 33/56983; G01N 2333/165; A61M 2202/0464; G01N 2333/8139; G01N 33/6854

(57) ABSTRACT

Disclosed herein, are compositions, methods, systems, and apparatus for collecting biological material from the nasal cavity of a subject or the olfactory region of a subject's nasal cavity. In some embodiments, the biological material is collected from a targeted sub-region of the nasal cavity, such as a targeted sub-region of the olfactory region, wherein such biological material is specific to the targeted sub-region. In some embodiments, the biological material collected from a targeted sub-region is preserved according to its localization. In some embodiments, the biological material comprises cerebrospinal fluid, one or more microbes of the patient's microbiome, one or more components of the patient's metabolome, one or more pathogens, and/or one or more biomarkers of interest. In some embodiments, a specific formulation is delivered to the region in the nasal cavity for facilitating biological material collection located therein.

### CROSS-REFERENCE

This application is a continuation of International Application No. PCT/IB2020/000293, filed Apr. 23, 2020, which claims the benefit of U.S. Provisional Application No. 62/838,169, filed Apr. 24, 2019, which are incorporated herein by reference in its entirety.

### BACKGROUND OF THE INVENTION

The human nasal cavity is a host for various types of microbes, which may be uniquely located in specific regions of the nasal cavity. The geometry and heterogeneity of the nasal cavity provides various features that result in such distribution of microbes across different locations. Other types of biological material are also found within the nasal cavity.

### SUMMARY OF THE INVENTION

In one aspect, provided herein, is a method of collecting biological material from an olfactory region of a patient, comprising: a) providing a formulation configured to capture the biological material; b) inserting a delivery device comprising a delivery orifice into a nasal cavity of the patient; c) delivering the formulation via the delivery device into the olfactory region or a targeted sub-region of the olfactory region of the patient; d) allowing the formulation to capture the biological material; and, e) withdrawing at least a portion of the formulation and the biological material captured therein, thereby collecting the biological material. In some embodiments, the delivery orifice is positioned such that the delivery of the formulation is to the targeted sub-region of the olfactory region. In some embodiments, the method further comprising preserving the composition of the formulation and/or captured biological material when being withdrawn. In some embodiments, the biological material comprises cerebrospinal fluid, one or more microbes of the patient's microbiome, one or more components of the patient's metabolome, one or more pathogens, and/or one or more biomarkers of interest. In some embodiments, the one or more pathogens comprise a virus or a portion or derivative thereof. In some embodiments, the virus is SARS CoV-2. In some embodiments, the delivery device comprises a cannula and/or microfluidic channel, and wherein inserting the delivery device comprises inserting the cannula and/or microfluidic channel into the nasal cavity of the patient. In some embodiments, the method further comprising determining a length of the patient's nasal cavity from the nostril to the olfactory region and inserting the cannula and/or microfluidic channel to a pre-determined depth based on the determined length. In some embodiments, the method further comprising placing a reference device on the face of the patient so as to provide an anatomical reference point for accurate placement of the delivery orifice into the nasal cavity. In some embodiments, the biological material is captured from the targeted sub-region of the olfactory region. In some embodiments, the delivery device comprises a sheath configured to minimize or prevent contamination of the cannula and/or microfluidic channel, the delivery orifice, the formulation and/or the captured biological material from non-olfactory regions of the nasal cavity, and/or from regions of the olfactory region other than the targeted sub-region of the olfactory region. In some embodiments, the sheath comprises a protective coating disposed about the cannula and/or microfluidic channel. In some embodiments, the sheath comprises a cover or sleeve disposed about the cannula and/or microfluidic channel. In some embodiments, the method further comprising inducing the patient so as to increase mucous production or decrease mucous production to facilitate the capture and/or collection of the biological material. In some embodiments, the method further comprising inducing the patient so as to increase blood flow or decrease blood flow to facilitate the capture and/or collection of the biological material. In some embodiments, the method further comprising inducing the patient so as to increase intracranial pressure to facilitate the capture and/or collection of the biological material. In some embodiments, the method further comprising applying energy to facilitate the capture and/or collection of the biological material. In some embodiments, applying energy comprises applying heat to the formulation through UV/VIS/IR light, through ohmic heating of the formulation, or through conduction from a heated element within the delivery device. In some embodiments, electric and/or magnetic fields are applied to facilitate the capture of the biological material by the formulation. In some embodiments, the delivery device is configured to deliver a flow of formulation to the olfactory region or targeted sub-region of the olfactory region, such that the flow of formulation is withdrawn as a continuous flow. In some embodiments, the method further comprising repeating the method so as to increase the collection of the biological material. In some embodiments, the formulation is of any formulation disclosed herein, including as disclosed in paragraph [008].

In another aspect, provided herein, is a method of collecting biological material from a nasal cavity of a patient, comprising: a) providing a formulation configured to capture the biological material; b) inserting a delivery device comprising a delivery orifice into the nasal cavity or a targeted subregion of the nasal cavity of the patient; c) delivering the formulation via the delivery device into the nasal cavity of the patient; d) allowing the delivered formulation to capture the biological material; and, e) withdrawing at least a portion of the formulation and the biological material captured therein. In some embodiments, the delivery orifice is positioned such that the delivery of the formulation is to the targeted sub-region of the nasal cavity. In some embodiments, the method further comprising preserving the composition of the formulation and/or captured biological material when being withdrawn. In some embodiments, the biological material comprises cerebrospinal fluid (CSF), one or more microbes of the patient's microbiome, one or more components of the patient's metabolome, one or more pathogens, and/or one or more biomarkers of interest. In some embodiments, the one or more pathogens comprise a virus or a portion or derivative thereof. In some embodiments, the virus is SARS CoV-2. In some embodiments, the delivery device comprises a cannula and/or microfluidic channel, and wherein inserting the delivery device comprises inserting the cannula and/or microfluidic channel into the nasal cavity of the patient. In some embodiments, the method further comprising determining a length of the patient's nasal cavity from the nostril to the olfactory region and inserting the cannula and/or microfluidic channel to a pre-determined depth based on the determined length. In some embodiments, the method further comprising placing a reference device on the face of the patient so as to provide an anatomical reference point for accurate placement of the delivery orifice into the nasal cavity. In some embodiments, the biological material is captured from a targeted region of the nasal cavity. In some embodiments, the delivery device comprises a sheath configured to minimize or prevent contamination of the cannula and/or microfluidic channel, the delivery orifice, the formulation and/or the captured biological material from a non-targeted region of the nasal cavity. In some embodiments, the sheath comprises a protective coating disposed about the cannula and/or microfluidic channel. In some embodiments, the sheath comprises a cover or sleeve disposed about the cannula and/or microfluidic channel. In some embodiments, the method further comprising inducing the patient so as to increase mucous production or decrease mucous production to facilitate the capture and/or collection of the biological material. In some embodiments, the method further comprising inducing the patient so as to increase blood flow or decrease blood flow to facilitate the capture and/or collection of the biological material. In some embodiments, the method further comprising inducing the patient so as to increase intracranial pressure to facilitate the capture and/or collection of the biological material. In some embodiments, the method further comprising applying energy to facilitate the capture and/or collection of the biological material. In some embodiments, applying energy comprises applying heat to the formulation through UV/VIS/IR light, through ohmic heating of the formulation, or through conduction from a heated element within the delivery device. In some embodiments, electric and/or magnetic fields are applied to facilitate the capture of the biological material by the formulation. In some embodiments, the delivery device is configured to deliver a flow of the formulation to the nasal cavity or targeted sub-region of the nasal cavity, such that the flow of formulation is withdrawn as a continuous flow. In some embodiments, the method further comprising repeating the method so as to increase the collection of the biological material. In some embodiments, the formulation is of any formulation disclosed herein, including as disclosed in paragraph [008].

In another aspect, provided herein, is an apparatus for collecting biological material from a nasal cavity of a patient, comprising: a) a first body containing a formulation configured to capture the biological material; b) a first cannula and/or a microfluidic channel comprising a delivery orifice configured for positioning in the nasal cavity of the patient and fluidly connected to the first body; c) a deployment mechanism for delivering the formulation through the first cannula and/or microfluidic channel into the nasal cavity of the patient, so as to capture biological material from the nasal cavity of the patient; and d) a collection device for collecting the captured biological material from the nasal cavity of the patient. In some embodiments, the delivery orifice is configured such that the delivery of the formulation is to the targeted sub-region of the nasal cavity. In some embodiments, the delivery orifice is configured such that the delivery of the formulation is to an olfactory region of the nasal cavity. In some embodiments, the delivery orifice is configured such that the delivery of the formulation is to a targeted sub-region of the olfactory region. In some embodiments, the biological material comprises cerebrospinal fluid (CSF), one or more microbes of the patient's microbiome, one or more components of the patient's metabolome, one or more pathogens, and/or one or more biomarkers of interest. In some embodiments, the one or more pathogens comprise a virus or a portion or derivative thereof. In some embodiments, the virus is SARS CoV-2. In some embodiments, the biological material is captured from the targeted sub-region of the nasal cavity. In some embodiments, the biological material is captured from an olfactory region of the nasal cavity. In some embodiments, the biological material is captured from a targeted sub-region of the olfactory region of the nasal cavity. In some embodiments, the apparatus further comprises a sheath configured to minimize or prevent contamination of the first body, the first cannula and/or microfluidic channel, the delivery orifice, the collection device, the formulation and/or the captured biological material from non-targeted regions of the nasal cavity. In some embodiments, the sheath is configured to minimize or prevent contamination of the first body, the first cannula and/or microfluidic channel, the delivery orifice, the collection device, the formulation and/or the captured biological material from non-targeted sub-regions of the olfactory region. In some embodiments, the sheath comprises a protective coating disposed about the first cannula and/or microfluidic channel. In some embodiments, the sheath comprises a cover or sleeve disposed about the first cannula and/or microfluidic channel. In some embodiments, the first body comprises a first container detachably coupled to the first cannula and/or microfluidic channel. In some embodiments, the collection device comprises a second body detachably coupled to the first cannula and/or microfluidic channel. In some embodiments, the collection device comprises a second body and a second cannula coupled to the second body. In some embodiments, the collection device is configured to preserve the integrity and biological material according to its localization as captured from the nasal cavity. In some embodiments, the deployment mechanism comprises a first actuator coupled to a first spring that is coupled to a first plunger. In some embodiments, the apparatus further comprises a clip configured to couple with the patient's nose so as to facilitate the positioning of the delivery orifice. In some embodiments, the first cannula and/or microfluidic channel is configured to move relative to the clip. In some embodiments, the collection device comprises a second actuator coupled to a second spring that is coupled to a second plunger. In some embodiments, the first body comprises a carpule. In some embodiments, the first cannula and/or microfluidic channel is a flexible cannula. In some embodiments, the first cannula and/or microfluidic channel is a telescoping cannula. In some embodiments, the apparatus comprises mechanical features to prevent hazardous forces being transmitted through the first cannula and/or microfluidic channel. In some embodiments, the mechanical features comprise a force limiting spring, a radial slip clutch, and/or an axial slip clutch. In some embodiments, the targeted sub-region of the olfactory region is localized to regions of discrete millimeters within the olfactory region. In some embodiments, the formulation is of any formulation disclosed herein, including as disclosed in paragraph [008].

In another aspect, provided herein, is a system for collecting biological material from a nasal cavity of a patient, comprising: a) a first body configured to contain a formulation; b) a first cannula and/or a microfluidic channel comprising a delivery orifice configured for positioning in the nasal cavity of the patient and fluidly connected to the first body; c) a deployment mechanism for delivering the formulation through the first cannula and/or microfluidic channel into the nasal cavity of the patient; d) a collection device for collecting the biological material from the nasal cavity of the patient; and e) the formulation, wherein the formulation is configured to capture the biological material. In some embodiments, the delivery orifice is configured such that the delivery of the formulation is to a targeted sub-region of the nasal cavity. In some embodiments, the delivery orifice is configured such that the delivery of the formulation is to an olfactory region of the nasal cavity. In some embodiments, the delivery orifice is configured such that the delivery of the formulation is to a targeted sub-region of the olfactory region. In some embodiments, the biological material comprises cerebrospinal fluid (CSF), one or more microbes of the patient's microbiome, one or more components of the patient's metabolome, one or more pathogens, and/or one or more biomarkers of interest. In some embodiments, the one or more pathogens comprise a virus or a portion or derivative thereof. In some embodiments, the virus is SARS CoV-2. In some embodiments, the biological material is captured from a targeted region of the nasal cavity. In some embodiments, the biological material is captured from an olfactory region of the nasal cavity. In some embodiments, the biological material is captured from a targeted sub-region of the olfactory region of the nasal cavity. In some embodiments, the system comprises a sheath configured to minimize or prevent contamination of the first body, the first cannula and/or microfluidic channel, the delivery orifice, the collection device, the formulation and/or the captured biological material from non-targeted regions of the nasal cavity. In some embodiments, the sheath is configured to minimize or prevent contamination of the first body, the first cannula and/or microfluidic channel, the delivery orifice, the collection device, the formulation and/or the captured biological material from non-olfactory regions of the nasal cavity or non-targeted sub-regions of the olfactory region. In some embodiments, the sheath comprises a protective coating disposed about the first cannula and/or microfluidic channel. In some embodiments, the sheath comprises a cover or sleeve disposed about the first cannula and/or microfluidic channel. In some embodiments, the first body comprises a first container detachably coupled to the first cannula and/or microfluidic channel. In some embodiments, the collection device comprises a second body detachably coupled to the first cannula and/or microfluidic channel. In some embodiments, the collection device comprises a second body and a second cannula coupled to the second body. In some embodiments, the collection device is configured to preserve the integrity and biological material according to its localization as captured from the nasal cavity. In some embodiments, the deployment mechanism comprises a first actuator coupled to a first spring that is coupled to a first plunger. In some embodiments, the apparatus further comprises a clip configured to couple with the patient's nose so as to facilitate the positioning of the delivery orifice. In some embodiments, the first cannula and/or microfluidic channel is configured to move relative to the clip. In some embodiments, the collection device comprises a second actuator coupled to a second spring that is coupled to a second plunger. In some embodiments, the first body comprises a carpule. In some embodiments, the first cannula and/or microfluidic channel is a flexible cannula. In some embodiments, the first cannula and/or microfluidic channel is a telescoping cannula. In some embodiments, the system comprises mechanical features to prevent hazardous forces being transmitted through the cannula. In some embodiments, the mechanical features comprise a force limiting spring, a radial slip clutch, and/or an axial slip clutch. In some embodiments, the targeted sub-region of the olfactory region is localized to regions of discrete millimeters within the olfactory region. In some embodiments, the formulation is of any formulation disclosed herein, including as disclosed in paragraph [008].

In another aspect, provided herein, is a method of making a diagnosis of a patient, comprising: a) performing the method of any one of claims 1-44, thereby collecting the biological material from the patient; b) analyzing the collected biological material; and c) based on the analysis of step b., making the diagnosis. In some embodiments, analyzing the biological material comprises identifying and/or quantifying biomarkers, pathogens, and/or microbes in the collected biological material. In some embodiments, the method further comprising correlating the identified and/or quantified biomarkers, pathogens, and/or microbes with a corresponding physiological characteristic and/or medical condition. In some embodiments, analyzing the biological material comprises using a point-of-care assay system. In some embodiments, the point-of-care assay system is configured to receive a sample of the collected biological material from the delivery device from a) any method disclosed herein, including as disclosed in paragraphs [003]-[004], b) any apparatus disclosed herein, including as disclosed in paragraph [005], or c) any system disclosed herein, including as disclosed in paragraph [006].

In another aspect, provided herein is a formulation for collecting biological material from a nasal cavity of a patient, wherein the formulation is configured to capture biological material once delivered within the nasal cavity, the delivered formulation configured to be withdrawn from the nasal cavity with the biological material. In some embodiments, the formulation is delivered to the olfactory region of the nasal cavity. In some embodiments, the formulation is configured to capture biological material from a targeted sub-region of the olfactory region. In some embodiments, the delivered formulation is configured to preserve the captured biological material when being withdrawn. In some embodiments, the biological material comprises cerebrospinal fluid (CSF), one or more microbes of the patient's microbiome, one or more components of the patient's metabolome, one or more pathogens, and/or one or more biomarkers of interest. In some embodiments, the formulation is configured to capture specific biological material. In some embodiments, the formulation comprises a buffered saline solution. In some embodiments, the buffered saline solution is 100 mM phosphate buffered saline. In some embodiments, the formulation comprises one or more gelling agents and/or thickeners. In some embodiments, the formulation comprises a viscosity modifier to provide a desired viscosity for the formulation. In some embodiments, the viscosity modifier comprises at least one of glycerol, pectin, and polyethylene glycol. In some embodiments, the viscosity modifier comprises 25-75% of the formulation by volume. In some embodiments, the formulation has a higher osmolality than fluid in the nasal cavity, the olfactory region, or a targeted sub-region of the olfactory region of the patient. In some embodiments, the formulation has an osmolality equal to or less than fluid in the nasal cavity, the olfactory region, or a targeted sub-region of the olfactory region of the patient. In some embodiments, a desired osmolality of the formulation is achieved through inclusion of salts, sugars, starches, albumin, dextran, or combinations thereof in the formulation. In some embodiments, the delivered formulation has an osmolality adjusted such that said osmolality is equal to a targeted osmolality after a target volume of fluid other than the formulation has been withdrawn from the nasal cavity, olfactory region or the targeted sub-region of the olfactory region. In some embodiments, the osmolality of the formulation is configured to vary over time, so as to capture biological material from the nasal cavity, the olfactory region, or the targeted sub-region of the olfactory region at a desired rate. In some embodiments, the osmolality of the formulation is configured to vary over time by inclusion of osmotic modifying agents in the formulation. In some embodiments, the osmotic modifying agents comprise micro-encapsulated particles of one or more osmotic modifying agents. In some embodiments, the one or more osmotic modifying agents comprise sodium chloride. In some embodiments, the micro-encapsulated particles comprise an enteric coating containing the one or more osmotic modifying agents. In some embodiments, the enteric coating is configured to release the one or more osmotic modifying agents upon exposure to defined conditions for a defined time period. In some embodiments, the defined conditions comprise one or more conditions selected from the group consisting of a temperature range, a pH range, and a defined shear force. In some embodiments, the formulation comprises an agent that promotes mucus production within the nasal cavity, the olfactory region, or the targeted sub-region of the olfactory region, so as to facilitate the capture of the biological material. In some embodiments, the agent that promotes mucus production is capsaicin. In some embodiments, the formulation comprises one or more agents that thicken mucus within the nasal cavity, the olfactory region, or the targeted sub-region of the olfactory region, so as to prevent the delivered formulation from moving, thereby increasing residence time of the delivered formulation within the nasal cavity, the olfactory region or the targeted sub-region of the olfactory region. In some embodiments, the formulation is configured to change from a liquid state to a semi-solid state upon delivery to the nasal cavity, the olfactory region, or to the targeted sub-region of the olfactory region. In some embodiments, the formulation is configured to initiate a cross-linking reaction upon delivery to the nasal cavity, the olfactory region, or to the targeted sub-region of the olfactory region. In some embodiments, the formulation comprises two or more reagents. In some embodiments, the two or more reagents are configured to mix upon delivery to the nasal cavity, the olfactory region, or to the targeted sub-region of the olfactory region, so as to initiate the cross-linking reaction, thereby changing the formulation into a semi-solid state. In some embodiments, the formulation comprises a non-Newtonian fluid. In some embodiments, the formulation changes from a liquid state to a semi-solid state at a temperature of about that of human body temperature. In some embodiments, the formulation changes from a liquid state to a semi-solid state at a temperature of about 35° C. to about 40° C. In some embodiments, the formulation changes from a liquid state to a semi-solid state at a temperature of about 37° C. In some embodiments, the formulation comprises a Bingham plastic. In some embodiments, the formulation behaves as a liquid when subject to shear force during delivery to the nasal cavity, the olfactory region, or to the targeted sub-region of the olfactory region. In some embodiments, the formulation behaves as a semi-solid when not subject to shear force. In some embodiments, the formulation further comprises a tail formed through the delivery and partial solidification of the formulation. In some embodiments, the tail is configured to be mechanically removed, thereby facilitating removal of the captured biological material. In some embodiments, the semi-solid state of the formulation is configured to preserve the captured biological material according to its localization. In some embodiments, the formulation acts as a carrier formulation. In some embodiments, the carrier formulation comprises encapsulated nano-particles. In some embodiments, the encapsulated nano-particles are encapsulated in a coating that breaks down upon exposure to defined conditions for a defined time period. In some embodiments, the defined conditions are unique to the nasal cavity, olfactory region, or targeted sub-region of the olfactory region. In some embodiments, the defined conditions comprise temperature, pH, and/or contact with a specific biological material. In some embodiments, the breakdown of the coating releases a chemical configured to change the carrier-formulation from a semi-solid state to a liquid state. In some embodiments, the formulation comprises one or more specific mono or polyclonal antibodies so as to target a specific biological material. In some embodiments, the formulation comprises one or more specific aptamers so as to target a specific biological material. In some embodiments, the specific biological material is cystatin-C. In some embodiments, the specific biological material is a virus or a portion or derivative thereof. In some embodiments, the virus is SARS CoV-2. In some embodiments, the formulation comprises anti-microbial agents so as to preserve the captured biological material. In some embodiments, the anti-microbial agents comprise 25% v/v ethanol and/or 5% w/v citric acid. In some embodiments, the formulation comprises microbial enriching and preservation material. In some embodiments, the microbial enriching and preservation material comprises 25% v/v tryptic soy broth. In some embodiments, the formulation comprises hydrogels. In some embodiments, the formulation comprises sugar. In some embodiments, the formulation is shear thinning or shear thickening. In some embodiments, the formulation is immiscible with water. In some embodiments, the formulation is miscible with water. In some embodiments, the formulation is configured to preserve the biological material. In some embodiments, the formulation is configured to preserve an integrity of the biological material. In some embodiments, the formulation is configured to change into a cohesive body after delivery to the nasal cavity, the olfactory region, or the targeted sub-region of the olfactory region. In some embodiments, the formulation comprises a solvent that evaporates to change the formulation into a cohesive body. In some embodiments, the formulation comprises a chemical agent that a) reacts after a time delay, b) reacts with air, c) reacts with a separately introduced gas or liquid, or d) reacts with a patient's body fluid, so as to form a cohesive body. In some embodiments, the formulation is configured to absorb the biological material from the olfactory region. In some embodiments, the formulation is provided, delivered, and/or withdrawn as a bolus of the formulation.

In some embodiments, method, apparatus and system comprise a robust novel nasal microbiome sampling system capable of collecting and preserving captured biological material from the olfactory region distinct from the lower nasal cavity geography. In some embodiments, the device is a class II diagnostic device, wherein the device would facilitate targeted sampling of the olfactory region. In some embodiments, the device comprises a telescoping sampling cannula that is sheathed and delivers a specialized formulation configured for preserving the biological material, including microbes of the microbiome, according to its localization for further analysis.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 depicts a flow chart showing the steps in a method for collecting biological material according to one embodiment.

FIG. 2A depicts an illustration of an embodiment for collecting biological material from a nasal cavity, wherein the embodiment comprises a device comprising a cannula that is inserted into the nasal cavity.

FIG. 2B depicts an illustration of the embodiment for collecting biological material according to FIG. 2A, wherein a container holding formulation is coupled to the cannula.

FIG. 2C depicts an illustration of the embodiment for collecting biological material according to FIG. 2A, wherein the formulation is delivered to the nasal cavity.

FIG. 2D depicts an illustration of the embodiment for collecting biological material according to FIG. 2A, wherein biological material is captured by the formulation.

FIG. 2E depicts an illustration of the embodiment for collecting biological material according to FIG. 2A, wherein a recovery vessel is coupled to the cannula to withdraw and collect the biological material.

FIG. 2F depicts an illustration of the embodiment for collecting biological material according to FIG. 2A, wherein the formulation and captured biological material is withdrawn through the cannula and into the recovery vessel.

FIG. 3A depicts an illustration of another embodiment for collecting biological material from a nasal cavity, wherein the embodiment comprises a device comprising a flexible bulb, and a cannula that is inserted into the nasal cavity.

FIG. 3B depicts an illustration of the embodiment for collecting biological material according to FIG. 3A, wherein the formulation is delivered to the nasal cavity by pressing on the flexible bulb,

FIG. 3C depicts an illustration of the embodiment for collecting biological material according to FIG. 3A, wherein biological material is captured by the formulation,

FIG. 3D depicts an illustration of the embodiment for collecting biological material according to FIG. 3A, wherein the flexible bulb is allowed to relax to withdraw and capture the biological material.

FIG. 4A depicts an illustration of another embodiment for collecting biological material from a nasal cavity, wherein the embodiment comprises a delivery device comprising a container holding the formulation, a deployment mechanism, and a cannula that is inserted into the nasal cavity.

FIG. 4B depicts an illustration of the embodiment for collecting biological material according to FIG. 4A, wherein the formulation is delivered to the nasal cavity by pressing on the deployment mechanism.

FIG. 4C depicts an illustration of the embodiment for collecting biological material according to FIG. 4A, wherein the delivery device is removed from the nasal cavity.

FIG. 4D depicts an illustration of the embodiment for collecting biological material according to FIG. 4A, wherein biological material is captured by the formulation.

FIG. 4E depicts an illustration of the embodiment for collecting biological material according to FIG. 4A, wherein a recovery device and recovery cannula, filled with wicking material, is inserted into the nasal cavity.

FIG. 4F depicts an illustration of the embodiment for collecting biological material according to FIG. 4A, wherein the recovery device draws the formulation and biological material through the cannula using the wicking material.

### DETAILED DESCRIPTION OF THE INVENTION

Biological material found in the nasal cavity may include biomarkers, pathogens, microbes of the human microbiome, and other material that provide information relating to the health and/or condition of a person. Disclosed herein, are compositions, methods, systems, and apparatus for collecting biological material from the nasal cavity of a person. In some embodiments, the biological material is collected from the olfactory region of the nasal cavity. In some embodiments, the biological material is collected from a targeted sub-region of the olfactory region, wherein such biological material is unique and distinct from other sub-regions of the olfactory region, and other non-olfactory regions of the nasal cavity. In some embodiments, the biological material collected from a targeted sub-region is preserved according to its localization. In some embodiments, the biological material comprises cerebrospinal fluid, microbes of a person's microbiome, metabolome, pathogens, and biomarkers of interest. In some embodiments, a specific formulation is delivered to the region in the nasal cavity for facilitating biological material collection located therein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term biological material as used herein refers to material produced by a living organism, and includes cerebrospinal fluid (CSF), one or more microbes of a patient's microbiome, biomarkers, sub-combination of biomarkers, one or more pathogens, one or more components of a patient's metabolome, other components, or any combination thereof.

The term formulation, as used herein, refers to compositions configured to capture biological material from the nasal cavity, including the olfactory region and targeted sub-regions within the olfactory region.

The term nasal cavity, as disclosed herein, comprises at least a lower nasal cavity, a middle nasal cavity, and an upper nasal cavity, wherein the upper nasal cavity comprises at least an olfactory region.

The term olfactory region refers to an area on and above the superior conchae and on the adjoining nasal septum where the mucous membrane has olfactory epithelium and olfactory glands.

The term target site, as used herein, refers to a desired location within the nasal cavity for capturing biological material. The desired location within the nasal cavity includes the olfactory region, the lower nasal cavity, the middle nasal cavity, and/or a targeted sub-region of the olfactory region.

The term targeted sub-region, as used herein, refers to a specific region of the nasal cavity, such as a specific region of the olfactory region and/or a non-olfactory region, where specific biological material is located.

The term biomarker, as used herein, refers to a characteristic that is objectively measured as an indicator of normal biological processes, pathogenic processes, or pharmacological responses to therapeutic intervention. Biomarker, as used herein, may refer to proteins, peptides, small molecules, and nucleic acids, microbes, the presence and/or concentration of which are suggestive or diagnostic of a disease or other medical condition, or indicate some biochemical imbalance within the brain.

The term CSF refers to cerebrospinal fluid.

The term microbiome, as used herein refers to the collection of microorganisms (such as bacteria, fungi, and viruses) within a particular environment, especially the collection of microorganisms living in or on the human body.

The term sample, as used herein, refers to biological material captured from a specific location of a patient. Sample, as used herein, may refer to biological material retrieved from within the nasal cavity, including specific regions within the nasal cavity, such as the olfactory region of the nasal cavity.

The term olfactory sample, as used herein is a sample comprising biological material retrieved from the olfactory region of the nasal cavity that contains microbiome, metabolome, CSF, other biomarkers of interest and any sub-combination of biomarkers or other components thereof.

The term sampling, as used herein, refers to collecting a sample of biological material from a specific location of a patient. For example, olfactory sampling refers to collecting a sample of biological material from the olfactory region.

The term capture is interchangeably used with acquire and retrieve. As used herein, the term capture (capturing) refers to biological material obtained from a target site by the formulation.

The term collect (collecting) as used herein refers to withdrawing captured biological material (with or without a corresponding formulation) from a target site.

In some embodiments, the compositions, methods, systems and apparatus described herein provide for minimally invasive and user- and patient-friendly biological material collection and analysis that can be used to diagnose a wide variety of infectious diseases affecting the brain and spinal cord, including but not limited to cancers, autoimmune disorders and central nervous system trauma. In some embodiments, apparatus and systems according to the present disclosure provide platforms that integrate, automate, and miniaturize the collection, processing, and analysis of biological material from a nasal cavity, including the olfactory region of the nasal cavity. Certain embodiments described herein allow researchers, clinicians and first responders to collect biological material (and/or biomarkers contained therein) in a minimally invasive and timely manner to advance treatment and resilience in neurological health and optimize human performance.

As described in International Patent Application No. PCT/CA2019/050455 filed Apr. 12, 2019, which is hereby incorporated herein by reference in its entirety, targeted drug delivery and precise bolus localization can be achieved with a minimally invasive cannula and delivery utilizing laminar flow and the coanda effect. Utilizing this approach, the dose fluid volume adheres to the superior aspect of the nasal corridor, accounting for anthropometric variability, and mitigating the need for operator adjustment and size-specific catheters. Devices according to PCT/CA2019/050455 can be used for collection of biological material in methods according to the present disclosure to allow for intuitive use, with low training requirements, and may provide devices with a small and light form factor that require no auxiliary devices for administration.

Recent in vivo dynamic PET scans have shown that the human nasal turbinate is a substantial part of the CSF clearance system (De Leon, M. J., Li, Y., Okamura, N., Tsui, W. H., Saint-Louis, L. A., Glodzik, L., . . . & Fossati, S. (2017). Cerebrospinal fluid clearance in Alzheimer disease measured with dynamic PET. Journal of Nuclear Medicine, 58(9), 1471). Assuming a CSF formation rate of 0.3 mL/min (Spector, R., Snodgrass, S. R., & Johanson, C. E. (2015). A balanced view of the cerebrospinal fluid composition and functions: focus on adult humans. Experimental neurology, 273, 57-68)] and a potential range of 1-20% uptake via the lymphatics near the cribriform plate (Sun, B. L., Wang, L. H., Yang, T., Sun, J. Y., Mao, L. L., Yang, M. F., . . . & Yang, X. Y. (2018). Lymphatic drainage system of the brain: A novel target for intervention of neurological diseases. Progress in neurobiology, 163, 118-143), a range of 3-60 μL/min of CSF in the nasal lymphatics is available for sampling by the methods and apparatus described herein.

The present disclosure provides compositions, referred to herein as formulations, configured for collecting biological material from the nasal cavity. In some embodiments, the formulations are configured for collecting biological material specific to the olfactory region. In some embodiments, the formulations are configured to inhibit or enhance recovery of certain biomarkers within collected biological material, as discussed below. By way of non-limiting example, a number of protein biomarkers of diagnostic interest may be found in the collected biological material, specifically if that sample contains components of cerebrospinal fluid (CSF), as set forth in Table 1 below:

TABLE 1
Exemplary Protein Biomarkers Found in Biological Material

| | | | | | Minimum | Maximum | | |
|---|---|---|---|---|---|---|---|---|
| | | | | | amount in | amount in | | |
| | Concentration in CSF (ng/mL) | | | | 50 μL for | 50 μL for | | |
| Protein | Healthy | | Disease | | disease | disease | | |
| Name | Low | High | Low | High | state (ng) | state (ng) | Disease marker | Notes |
| Cystatin-C | 2500 | 6500 | 1300 | 5800 | 65 | 290 | Amyotrophic | |
| | | | | | | | Lateral Sclerosis | |
| Alpha- | 52 | 85.5 | 38 | 70 | 1.9 | 3.5 | Parkinson's | |
| synuclein | | | | | | | Disease | |
| Apolipo- | 1800 | 7200 | 2100 | 4700 | 105 | 235 | Alzheimer's | Plays a role in |
| protein E | | | | | | | Disease | regeneration in |
| | | | | | | | | the brain |
| Ferritin light | 5.75 | 7.61 | 40.63 | 45.99 | 2.0315 | 2.2995 | Non- | Progressive biomarker |
| chain | | | | | | | inflammatory | for ALS, and for |
| | | | | | | | neurological | neuroferritinopathy |
| | | | | | | | disease (ie. | |
| | | | | | | | headaches, | |
| | | | | | | | cerebrospinal | |
| | | | | | | | leakage) | |
| | | | 111.15 | 118.41 | 5.5575 | 5.9205 | Amyotrophic | |
| | | | | | | | Lateral Sclerosis | |
| | | | — | <100 | 5 | 5 | Neuroferritinopa thy | |
| Hemopexin | 12300 | 32600 | 50000 | >50000 | 2500 | 2500 | Delayed cerebral | Binds to free heme |
| | | | | | | | ischemia after | |
| | | | | | | | subarachnoid | |
| | | | | | | | hemorrhage | |
| Ig kappa | 4 | 25.8 | 377.9 | 1340.9 | 18.895 | 67.045 | Multiple | Healthy control values |
| chain V-IV | | | | | | | sclerosis | refer to non- |
| region | | | | | | | | inflammatory |
| | | | | | | | | CNS reaction |
| Lactotrans- | undetect- | undetect- | 128 | 99430 | 6.4 | 4971.5 | Bacterial | Distinguishes between |
| ferrin | able | able | | | | | meningitis | aseptic or bacterial |
| | | | | | | | | meningitis in |
| | | | | | | | | children |
| | | | 0 | 2715 | 0 | 135.75 | Aseptic | |
| | | | | | | | meningitis | |
| Ig | 10 | 1000 | 1000 | 50000 | 50 | 2500 | Multiple sclerosis | Kappa/gamma ratio |
| kappa/ | | | | | | | (kappa | shows progression in |
| gamma | | | | | | | values) | Multiple Sclerosis, |
| | | | | | | | | healthy control values |
| | | | | | | | | refer to other |
| | | | | | | | | neurological |
| | | | | | | | | diseases |
| Glutathione | 10 | 1000 | 50 | 10000 | 2.5 | 500 | gamma values | |
| peroxidase 3 | 0.9 | 2.7 | 28.4 | 44.2 | 1.42 | 2.21 | Bacterial | U/mL. Levels observed |
| | | | | | | | meningitis | in children. Lower |
| | | | | | | | | levels also observed in |
| | | | | | | | | Parkinson's patients |
| | | | | | | | | but exact |
| | | | | | | | | concentrations |
| | | | | | | | | not reported. |
| | | | 2.6 | 4.6 | 0.13 | 0.23 | Aseptic | |
| | | | | | | | meningitis | |
| Cholinesterase | 0.018 | 0.21 | 0.157 | 0.0177 | 0.00785 | 0.000885 | Alzheimer's | U/mL. |
| | | | | | | | Disease | |
| Fibrinogen | 269 | 785 | 0 | 1898 | 0 | 94.9 | Major | Candidate marker for |
| gamma | | | | | | | Depressive | Alzheimer's; marker for |
| chain | | | | | | | Disorder | major depressive |
| | | | | | | | | disorder (MDD) |
| Gamma- | 1.2 | 3.2 | 2 | 10 | 0.1 | 0.5 | Febrile seizures | Diagnosis of |
| enolase | | | | | | | | Creutzfeldt- |
| | | | | | | | | Jakob disease |
| | | | 2 | 7.4 | 0.1 | 0.37 | Alzheimer's | |
| | | | | | | | Disease | |
| | | | 2 | 7 | 0.1 | 0.35 | Vascular | |
| | | | | | | | dementia | |
| Osteopontin | 142 | 430 | 29 | 370 | 1.45 | 18.5 | Multiple | |
| | | | | | | | sclerosis | |
| | | | 253 | 531 | 12.65 | 26.55 | Inflammatory | |
| | | | | | | | CNS disease | |
| | | | 22 | 191 | 1.1 | 9.55 | Glioblastoma | |
| C-reactive | 68400 | 108400 | 172500 | 742500 | 8625 | 37125 | Pyogenic | Values observed in |
| protein | | | | | | | meningitis | children. |
| | | | 0 | 470900 | 0 | 23545 | Partially treated | |
| | | | | | | | meningitis | |
| | | | 0 | 49900 | 0 | 2495 | Tubercular | |
| | | | | | | | meningitis | |
| | | | 0 | 21400 | 0 | 1070 | Viral meningitis | |
| | | | 7900 | 13900 | 395 | 695 | Tubercular | Values observed in |
| | | | | | | | meningitis | adults. |
| | | | 132200 | 407800 | 6610 | 20390 | Pyogenic | |
| | | | | | | | meningitis | |

Hemopexin is a protein that binds to free heme and its presence at >50,000 ng/mL is a predictor of cerebral ischemia after subarachnoid hemorrhage. In a 50 μL sample this is equivalent to >2,500 ng of analyte present.

C-Reactive Protein is a marker for diagnosing pyogenic meningitis. The healthy range is between 3,420-5,420 ng in a 50 μL sample. An increased 8,625-37,125 ng in a 50 μL sample is an indicator of meningitis in children, while a range of 6,610-20,310 ng in a 50 μL sample is an indicator for adults. It can also identify tubercular meningitis which is decreased, falling into a range of 0-2,495 ng in children, and 395-695 ng in adults.

Cystatin-C is a potential biomarker for Amyotrophic Lateral Sclerosis (ALS) Reduced levels of 65-290 ng in a 50 μL sample are indicative of the disease, compared to healthy levels of 125-325 ng in a 50 μL.

Certain embodiments provide CSF sampling formulations and apparatus that enhance the collection of CSF in comparison to other components in normal nasal discharge. Table 2 provides some factors that differentiate CSF from normal nasal discharge:

TABLE 2
Comparison Between CSF and Normal Nasal Discharge

| CSF | Normal Nasal Discharge |
|---|---|
| thin | thick |
| watery | mucoid |
| does not tend to stick to filter | tends to stick to filter membrane |
| membrane | negative for beta transferrin |
| positive for beta transferrin | not salty |
| salty, positive halo sign | negative halo sign |

Target sign (Oh J W, Kim S H, Whang K. Traumatic Cerebrospinal Fluid Leak: Diagnosis and Management. Korean J Neurotrauma. 2017; 13(2):63-67): When the CSF is mixed with a blood or nasal discharge, the CSF moves away on the filter paper, and the blood moves closer, so two rings are visible. This is called a target sign, a double ring sign, or a Halo sign. Embodiments of the device can utilize this to isolate CSF (i.e. stacked membrane filters in a sample collection reservoir to isolate the CSF from any blood present). Filter materials include natural cotton or synthetic fibers (such as polyester). Suitable materials include an adaptation of materials commonly used in lateral flow devices (like pregnancy test). Preferred materials include: natural cotton fibers, treated polyester fibers, nitrocellulose membranes, or polycarbonate mesh.

Binding test (Oh J W, Kim S H, Whang K. Traumatic Cerebrospinal Fluid Leak: Diagnosis and Management. Korean J Neurotrauma. 2017; 13(2):63-67): When the discharge from the nose is passed through a dry adsorbent woven material (i.e. dry gauze), the CSF is more likely to be clear if it is not sticky. This step is a test to determine the nasal discharge, which is unclear and sticky due to mucin secretion from the nose. Suitable materials include an adaptation of materials commonly used in lateral flow devices (like pregnancy test). Preferred materials include: natural cotton fibers, treated polyester fibers, nitrocellulose membranes, or polycarbonate mesh.

Glucose oxidized test: The CSF glucose from nasal or ear secretions has long been a classical method in testing for a CSF leak. In general, the glucose oxidase strips show a positive result when the sample has a concentration over 20 mg/dL. Nasal discharge has a normal concentration of 10 mg/dL of glucose, thus, if the glucose test is negative then it can be ruled out. However, it is only to be used as reference as it has high false positive and negative rates depending on the patients' other medical conditions. Moreover, the lacrimal secretion can also be tested even if the concentration is less than 5 mg/dL. Meanwhile, a false positive result can be observed in the bloody nasal discharge whereas false negative results are seen if the meningitis is already progressed in the patients. All these clinical conditions have to be considered before the interpretation and confirmation of the CSF leaks. Preferred embodiments may incorporate the glucose oxidase test strip in the sample reservoir or performed as an additional step to the sampling process.

Glucose and Chlorine Concentration: If the serum glucose level is 0.5 to 0.67 mg/dL, higher concentrations are suggestive of CSF. CSF glucose level is undoubtedly affected by the glucose levels in serum, therefore, it is important to consider the two parameters together when confirming the CSF detection. Samples with a Chlorine concentration level ≥100 mEq/L, is indicative of CSF. Preferred embodiments may incorporate the Glucose and Chlorine test strip in the sample reservoir or performed as an additional step to the sampling process to confirm CSF sampling.

Beta-2 Transferrin (Tau protein): Beta-1 transferrin is found in serum tears, nasal secretion and saliva ubiquitously while Beta-2-transferrin is only observed in CSF, perilymph, and vitreous humor. Since the Beta-2 transferrin is specific in CSF, it is a well-known marker with extremely high sensitivity and specificity. It is produced from transferrin by loss of sialic acid due to the presence of neuraminidase activity in the brain; therefore, beta-2 transferrin is located only within the CSF, perilymph, and aqueous humor. Its absence from other bodily secretions makes its detection invaluable in confirming the diagnosis of CSF rhinorrhea or otorrhea (leakage of CSF into the nose or ear canal, usually as a result of head trauma, tumor, congenital malformation or surgery)(Beta-2 Transferrin/Tau Protein: http://www.viapath.co.uk/our-tests/beta-2-transferrintau-protein). Tau protein, discovered in 1975, is an intraneuronal protein mainly involved in axonal transport and stabilization of microtubules. CSF Tau protein is a neuronal protein, commonly assessed for diagnosis of Alzheimer Disease (AD). An Enzyme Linked Immunosorbent Assay (ELISA) measurement of Tau protein in rhinorrhoea fluid may be a reliable and relevant marker for detecting the presence of CSF in the nasal discharge and sign the existence of a CSF leakage (Oudart J B, Zucchini L, Maquart F X, et al. Tau protein as a possible marker of cerebrospinal fluid leakage in cerebrospinal fluid rhinorrhoea: A pilot study. Biochem Med (Zagreb). 2017; 27(3):030703). Preferred embodiments may incorporate a lateral flow test strip with antibodies to TAU protein in the sample reservoir or performed as an additional step to the sampling process to confirm CSF sampling. Embodiments of the CSF sampling formulation may contain antibodies or other selective elements to selectively bind to samples containing TAU protein to enhance the selective recovery of CSF fluid.

Beta-Trace Protein (BTP): Also known as prostaglandin D synthase, this protein is synthesized primarily in arachnoid cells, oligodendrocytes, and the choroids plexus within the Central Nervous System (CNS). Beta-trace protein is also present in the human testes, heart, and serum. It is altered by the presence of renal failure, multiple sclerosis, cerebral infarction, and certain CNS tumors. This test has been used to diagnose CSF rhinorrhea in multiple studies, with a sensitivity of 92% and specificity of 100% (What is the role of beta-trace protein testing in the workup of cerebrospinal fluid (CSF) rhinorrhea? https://www.medscape.com/answers/861126-102445/what-is-the-role-of-beta-trace-protein-testing-in-the-workup-of-cerebrospinal-fluid-csf-rhinorrhea). BTP is a 25-kDa protein identified as prostaglandin D synthase. At almost 20 mg/L, it is the second-most abundant CSF protein after albumin, with a CSF-to-serum ratio of 33, the highest of all CSF-specific proteins (Bernasconi, Luca & Huber, Andreas. (2017). Beta-trace Protein Quantification for Diagnosis of CSF Leakage Syndrome. White Paper). Preferred embodiments may incorporate a lateral flow test strip with antibodies to detect BTP in the sample reservoir or performed as an additional step to the sampling process to confirm CSF sampling. Embodiments of the CSF sampling formulation may contain antibodies or other selective elements to selectively bind to samples containing BTP protein to enhance the selective recovery of CSF fluid.

For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

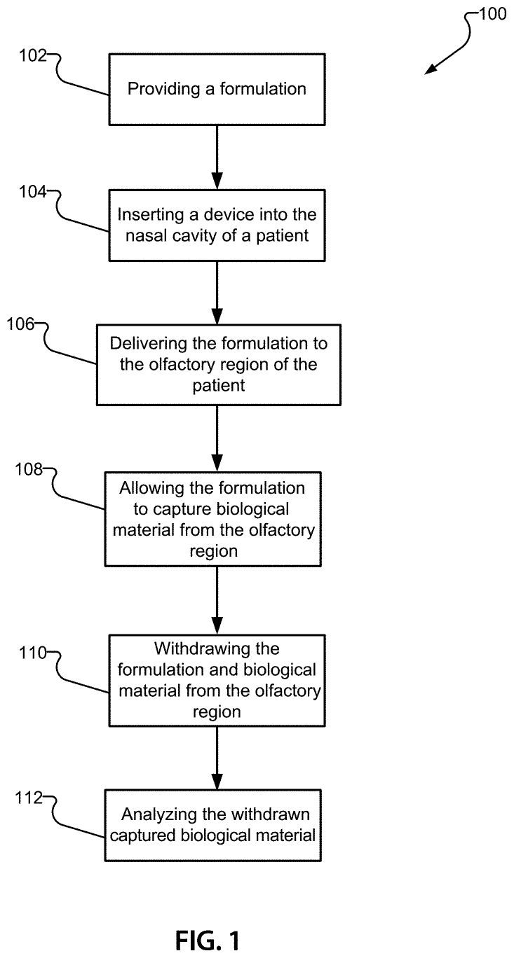

FIG. 1 depicts a flow chart for an exemplary method 100 to collect biological material from the nasal cavity of a person, or specifically, the olfactory region of the nasal cavity. The exemplary method as disclosed in FIG. 1 is also applicable to other regions of the nasal cavity, wherein reference to the olfactory region is substituted with another targeted region of the nasal cavity, or the nasal cavity itself. The method 100 comprises providing a formulation configured to target biological material from the nasal cavity (such as the olfactory region) at 102, inserting a formulation delivery device into the nasal cavity of a patient at 104, delivering the formulation to the olfactory region at 106, allowing the formulation to capture biological material from the olfactory region (including biomarkers of interest therein) at 108, withdrawing the formulation and captured biological material at 110, and analyzing the captured biological material, including biomarker(s) of interest therein, at 112. Each of these steps is described in detail in the following sections. In some embodiments, the withdrawn captured biological material (i.e. collected biological material) is preserved. In some embodiments, the withdrawn captured biological material is preserved according to its localization within the nasal cavity.

### Providing the Formulation

In some embodiments, exemplary method 100 includes providing a formulation at 102. In some embodiments, the formulation is configured to target biological material in a nasal cavity. In some embodiments, the formulation is configured for a target site within the nasal cavity. In some embodiments, the target site is the olfactory region, wherein the formulation is configured to target specific biological material, including biomarkers contained within the olfactory region. In some embodiments, such biological material include CSF, microbes from a person's microbiome, metabolome, and other biomarkers of interest). In some embodiments, the target site is a targeted sub-region of the nasal cavity, such as a targeted sub-region within the olfactory region, wherein formulation is configured to target specific biological material within the targeted sub-region. The following sections describe examples of formulations for nasal cavity sampling (nasal cavity sampling formulation), including formulations for olfactory sampling (olfactory sampling formulation), as well as examples of formulations configured to target specific biological material. As described herein, the term sampling refers to collecting a sample of biological material from a specific location, such as capturing and withdrawing biological material from the olfactory region (i.e. olfactory sampling). In some embodiments, the formulation, such as an olfactory sampling formulation, comprises a buffered saline solution. In some embodiments, the buffered saline solution comprises a viscosity modifier to provide a desired viscosity for the formulations. In some embodiments, the formulations comprise of one or more inactive ingredients that are FDA- or EMA-approved for nasal administration. The FDA Inactive Ingredient Database, and the Annex to the European Commission guideline on ‘Excipients in the labelling and package leaflet of medicinal products for human use’ are both incorporated herein by reference, for the purpose of providing examples of inactive ingredients approved for spray or aerosol dosage forms, and/or nasal and inhalation routes of administration.

In some embodiments, the formulation is held in a container that a) provides sufficient shelf life for product viability, b) integrates with an appropriate container filling line, and c) integrates with a dispensing system. For example, the formulation may be stored in a carpule (single or multi-chamber), syringe (single or multi-chamber), disposable pipette, pipette, bulb syringe, blow-fill-seal container (e.g. MicroDose™ single use unit or SwabDose™ single use unit), bellows, microfluidic cartridge, unit dose liquid cup, vial, ampule, heat sealed bag (e.g. IV bag), molded bag, or custom component assembly. Exemplary formulation are described below.

### Formulation Comprising Buffered Saline and Viscosity Modifier

In some embodiments, a formulation for nasal cavity sampling, including olfactory sampling, comprises 100 mM phosphate buffered saline that comprises an approved viscosity modifier such as glycerol ranging from 25-75% v/v. In some embodiments, the formulation comprises 100 mM phosphate buffered saline that comprises an approved viscosity modifier such as Pectin ranging from 25-75% v/v. In some embodiments, the formulation comprises 100 mM phosphate buffered saline that comprises an approved viscosity modifier such as polyethylene glycol 3350 ranging from 25-75% v/v.

In some embodiments, the formulation is immiscible with water. In some such embodiments, the formulation is delivered into the olfactory region to form a bolus. In these embodiments, the biological material, including microbes from the microbiome, metabolome, CSF, and/or biomarker(s) of interest diffuses from a target site in the olfactory region into the formulation bolus.

In some embodiments, the formulation is miscible with water, and is at a lower osmolality than the patient's fluids (e.g., mucus, CSF, plasma, blood, seroma fluid) at the target site within the olfactory region or other nasal cavity regions. For example, a range for the osmolality of plasma can be 275-299 milli-osmoles/Kg in healthy people, with a similar osmolality range for CSF (mean 270 milli-osmoles/Kg). Such osmolality ranges however change in disease state, and can vary across individuals. In some embodiments, the formulation is designed to be at a lower osmolality than the extreme low end of varying osmolality ranges to accommodate this sampling function across individuals.

In some embodiments, the formulation is miscible with water, and is at equal osmolality to the patient's fluids at the target site. In some such embodiments, liquid from the formulation couples to specific biomarkers of interest in the mucus layer of the patient, and the biomarker of interest diffuses into the formulation.

In some embodiments, the formulation is miscible with water and is at a higher osmolality than the patient fluids at the target site. In some such embodiments, liquid from the mucus layer of the patient is drawn into the formulation. In some embodiments, the osmolality of the formulation is adjusted to become equal with the osmolality of the target site after an appropriate volume of nasal cavity fluid has been extracted from the target site. In some embodiments, the osmolality in the formulation is adjusted by adding salts, sugars, starches, albumin, dextran, other agents, or combinations thereof. In some embodiments, suitable sugars include, but are not limited to, sucrose, glucose, dextrose, and sugar alcohols such as mannitol, xylitol, and the like.

In some embodiments, the formulation includes chemical compounds that control osmolality over time to ensure that biological material is drawn from the target site at an appropriate rate. For example, in some embodiments, the formulation comprises micro-encapsulated particles of saline or other osmotic modifying agents which are encapsulated in an enteric coating, which breaks down when exposed to certain conditions such as temperature, pH, shear force etc. In some embodiments, the coating of such micro-encapsulated particles break down upon being exposed to such conditions for a requisite time period.

In some embodiments, the formulation further comprises hydrogels to promote transport of the biological material into the formulation bolus.

In some embodiments, the formulation comprises compounds that enhance recovery of the biological material from the target site. For example, in some embodiments, the formulation comprises capsaicin or another agent configured to increase mucus production at the target site, which increases the flow of a biomarkers of interest into the formulation bolus. In some embodiments, the formulation comprises agents that thicken the mucus layer to prevent the formulation bolus from moving, thereby allowing better residence time to draw in the biological material.

In some embodiments, the formulation includes gelling agents, thickeners, or other agents to control its viscosity, make it shear thinning or shear thickening, or make it a Bingham plastic, to ensure the formulation stays at the target site during capture of the biological material.

### Formulation Comprising Crosslinking Reagents

In some embodiments, the formulation comprises two or more reagents that are mixed to initiate a crosslinking reaction. In some embodiments, the two or more reagents are contained within a delivery device. In some embodiments, the two or more reagents are mixed upon deployment into the nasal cavity. In some embodiments, upon mixing, the reagents crosslink to form a semi-solid state to allow longer retention and sampling time. In some embodiments, delivery of the formulation leaves a trail extending from the delivery location of the formulation (i.e. target site) that forms a semi-solid tail portion of the formulation. Upon completion of desired dwell time, the ‘tail’ of the semi-solid formulation bolus is mechanically removed containing preserved captured biological material, including biomarkers of interest therein. In some embodiments, the crosslinking formulation enables preservation of a biological material according to its localization, i.e. biological material collected from a target site are not mixed with biological material from other regions in the nasal cavity. In some embodiments, crosslinking of the formulation locks the captured biological material in place, thereby enabling preservation of a collected biological material according to its localization, when the formulation is disposed in a targeted region for precision biological material localization. In some embodiments, the multi part components (e.g., use of two or more reagents) allow for longer shelf life of the formulation and contour forming upon deployment of the formulation to allow for sampling across a large population of diverse anatomies.

### Formulation Using Non-Newtonian Fluids

In some embodiments, the formulation comprises non-Newtonian fluids, such that the formulation is liquid when stored at room temperature and when deployed, wherein the formulation becomes semi-solid at a temperature of about human body temperature, forming a semi-solid state to allow longer retention and sampling time. In some embodiments, the formulation becomes semi-solid at a temperature of about 37° C. In some embodiments, the formulation becomes semi-solid at a temperature of about 35° C. to about 40° C. In some embodiments, delivery of the formulation leaves a trail extending from the delivery location of the formulation (i.e. target site) that forms a semi-solid tail portion of the formulation. In some embodiments, upon completion of a desired dwell time, the ‘tail’ of the semi-solid formulation bolus is mechanically removed containing the captured biological material. In some embodiments, a single component formulation is required and the formulation contour forms upon deployment to allow for sampling across a large population of diverse anatomies. In some embodiments, the semi-solid state of the formulation enables preservation of the captured biological material according to its localization. In some embodiments, the semi-solid formulation locks the captured biological material in place, thereby enabling preservation of a captured biological material according to its localization, when the formulation is disposed in a targeted region for precision biological material localization.

Formulation Comprising Bingham Plastic Fluids

In some embodiments, the formulation comprises Bingham plastic fluids, whereby the deployment of the formulation under shear causes the formulation to behave as a liquid state and allows for easy deployment to target area. In some embodiments, once the formulation is located in the targeted site, the shear force is removed and the Bingham plastic fluid causes the formulation to revert back to a semi-solid state and solidifies to allow longer retention and sampling time. In some embodiments, upon completion of a desired dwell time, application of shear forces (for example, through suction) to the formulation causes the Bingham plastic to convert to liquid state to allow for easy removal and extraction. At least one advantage with this embodiment is that one single formulation is required, the formulation contour forms upon deployment to allow for sampling across a large population of diverse anatomies, and facilitate removal and sample handling for post sampling analysis through the liquid aspect of the sampling formulation. In some embodiments, the semi-solid state of the formulation enables preservation of captured biological material according to its localization. In some embodiments, the semi-solid formulation locks the captured biological material in place, thereby enabling preservation of a captured biological material according to its localization, when the formulation is disposed in a targeted region for precision biological material localization.

### Formulations Comprising any Combination of Aforementioned Examples

In some embodiments, the formulation comprises a combination of any of the above examples of a formulation, including any combination of using Bingham plastic, crosslinking reaction, and/or non-Newtonian fluids. In some embodiments, the formulation, comprising of any combination of the aforementioned examples, is deployed in a liquid state and solidifies at the target site. In some embodiments, the formulation comprising any of the aforementioned examples, acts as a carrier formulation. In some embodiments, the carrier formulation further comprises encapsulated nano-particles that are encapsulated in a coating that breaks down under certain conditions unique to the targeted area. In some embodiments, such conditions comprise temperature, pH, contact with a specific biomarker of interest. In some embodiments, the coating of such encapsulated particles break down upon being exposed to such conditions for a requisite time period. In some embodiments, the encapsulated nano-particles are tailored to breakdown under a variety of specific biological conditions, so as to reflect different desired biological states. In some embodiments, the breakdown of the coating releases a chemical designed to react with carrier formulation in its semi-solid state, causing it to revert to its liquid state. At least one advantage with these embodiments include contour forming upon deployment to allow for sampling across a large population of diverse anatomies and facilitating removal and sample handling for post sampling analysis facilitated through the liquid aspect of the sampling formulation.

### Formulations Comprising Configured to Target Specific Biological Material

In some embodiments, the formulations are configured to target specific biological material located at the target site. In some embodiments it is desirable to detect, preserve, isolate and/or enhance or limit recovery of certain biological material located at the target site. Examples of specific approaches are described below.

Some embodiments provide a specific biological material targeting sampling formulation comprising of formulations according to any of the examples discussed above, further comprising specific mono or polyclonal antibodies to a specific biological material of interest. In some embodiments, the specific biological material of interest is a virus or a portion or derivative of the virus. For example, in some embodiments, the virus is SARS CoV-2. In some embodiments, the specific biological material of interest is cystatin-C.

Some embodiments provide a specific biological material targeting sampling formulations comprising of formulations according to any of the examples discussed above, further comprising specific aptamers to a specific biological material of interest. In some embodiments, the specific biological material of interest is a virus or a portion or derivative of the virus. For example, in some embodiments, the virus is SARS CoV-2. In some embodiments, the specific biological material of interest is cystatin-C.

Some embodiments provide a formulation with anti-microbial properties to aid in biological material preservation, such as a specific biological material targeting sampling formulation according to any of the formulation examples discussed above further comprising antimicrobial agents. In some embodiments, antimicrobial agents include 25% v/v ethanol and/or 5% w/v citric acid.

Some embodiments provide a formulation with anti-microbial properties to aid in biological material preservation, such as a specific biological material targeting sampling formulation according to any of the formulation examples discussed above further comprising antimicrobial agents. In some embodiments, antimicrobial agents include beta lactam antibiotics to remove peptidoglycan containing microorganisms only.

Some embodiments provide a formulation to enrich for specific microorganisms, such as a specific biological material targeting sampling formulation according to any of the formulation examples discussed above further comprising specific microbial enriching and preservation materials, such as 25% v/v tryptic soy broth (e.g. for detection and culture of microbial meningitis).

As one of skill in the art will appreciate, other specific configurations of formulations including combinations of any or all of the features discussed in the examples above may be provided in other embodiments.

### Device Insertion

Method 100 further includes inserting a device into the nasal cavity at 104. In some embodiments, the device is inserted proximate to the olfactory region of a patient at 104. In some embodiments, the device comprises a flexible cannula, a container holding the formulation, wherein the container is fluidly connected to the cannula, and a deployment mechanism for forcing the formulation out of the container and through the cannula. Suitable nasal cannulas are well-known in the art. In some embodiments, the cannula is a telescoping cannula. In some embodiments, the cannula comprises a delivery orifice through which the formulation is discharged from the cannula.

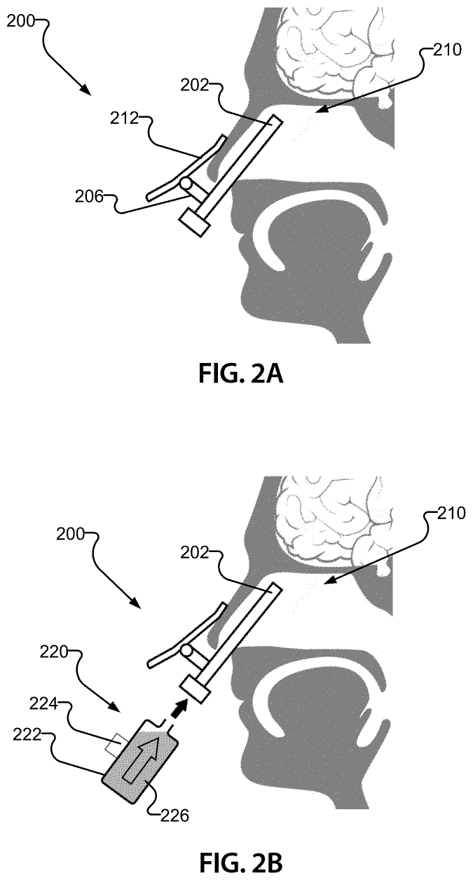

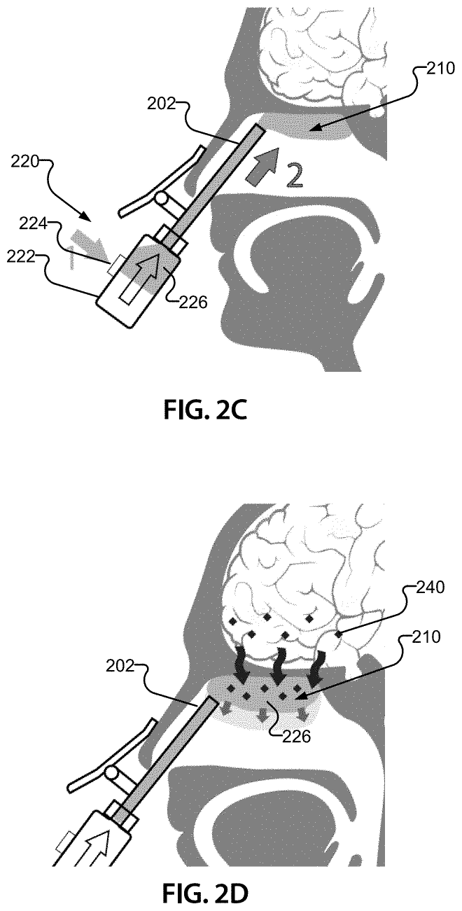

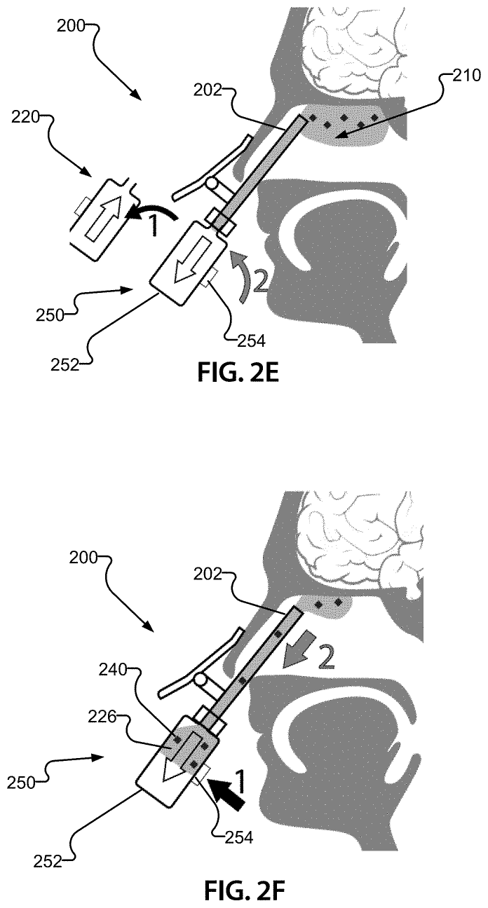

In some embodiments, the device includes features that locate the device on patient anatomy to enhance insertion safety and support self-administration. In some embodiments, the device includes features that locate against the external base of the nose and/or nostril, as discussed below with reference to FIG. 2a. In some embodiments, insertion of the device is done by feel/patient comfort.

In some embodiments, the device is located against an interior nasal anatomy (e.g., roof of the olfactory chamber below the cribriform plate or forward of the sphenoid sinus). For example, in some embodiments, the device includes mechanical features that prevent hazardous forces being transmitted through the cannula (e.g. a force limiting spring, a radial slip clutch, and/or an axial slip clutch). In one embodiment, the cannula and delivery system floats within the device and is attached to the device with a spring. In some embodiments, force from the body of the device is transmitted through the spring to the cannula. In some embodiments, when the cannula contacts the patient, the spring limits the maximum force that the device can transmit to the patient.

In other embodiments, the device is located against other external facial anatomy, for example, with reference to the bridge of the nose, cheek bones, below the eyebrows, or in front of the teeth. In another embodiment, the device is located on the face as a pair of eye glasses resting on the bridge of the nose.

In some embodiments, the device includes a sheath configured to prevent or minimize contamination of the cannula from non-targeted areas in the nasal cavity. In some embodiments, the sheath is configured to prevent or minimize biological material and/or formulation cross contamination with non-targeted areas of the nasal cavity. In some embodiments, wherein the target site is the olfactory region, non-targeted areas include the lower nasal cavity, middle nasal cavity, and regions of the upper nasal cavity other than the olfactory region. In some embodiments, wherein the targeted site is a targeted sub-region of the olfactory region, the non-targeted areas include the lower and middle nasal cavities and non-targeted sub-regions of the olfactory region.

### Delivery of Formulation

Method 100 further includes delivering the formulation through the device into the nasal cavity, such as the olfactory region of the patient at 106. In some embodiments, when deposited in the nasal cavity, the formulation is a formulation bolus. In some embodiments, the formulation is delivered using a cannula, or other microfluidic channel. In some embodiments, the formulation is delivered to a targeted sub-region of the nasal cavity or olfactory region. In some embodiments, the formulation is discharged from the device through an orifice of the cannula, wherein the orifice is positioned to deliver the formulation to the targeted sub-region. In some embodiments, the targeted sub-region is localized to regions of discrete millimeters within the olfactory region.

In some embodiments, the formulation is configured to move through the device to the olfactory region by a variety of mechanical methods, for example, by spring force, motor force, pneumatic pressure, vacuum, or force provided by the hand, driving the formulation out of a carpule (single, multi-chamber), syringe (single, multi-chamber), disposable pipette, pipette, bulb syringe, blow-fill-seal container (e.g. MicroDose™ single use unit), bellows, microfluidic cartridge, or molded bag. In some embodiments, the formulation is moved by a pump (e.g. peristaltic pump, piston pump, gear pump, etc.). In some embodiments, the formulation is moved by compressed gas provided by a pump (e.g. peristaltic pump, piston pump, gear pump, etc.) or by a reservoir of compressed gas (e.g. CO2 cylinder). In some embodiments, required force or pressure to move the formulation may be provided by an electric motor, voice coil, solenoid, or magnets. For example, in some embodiments, the formulation may be delivered using a device having features as disclosed in International Patent Application No. PCT/CA2019/050455.

In another embodiment, the patient exhales or inhales through the mouth into the device to provide pneumatic pressure or vacuum to drive the formulation out of the device and into the target site. In some embodiments, the device includes a mouthpiece that rests in the patient's mouth when the device is inserted in the nose. In some embodiments, the patient exhales into the mouth piece. In some embodiments, the applied pressure moves a piston which forces the formulation out of the device, through a cannula, and to the target site. In some embodiments, applied pressure further compresses a bag, or bellows filled with the formulation. In some embodiments, the patient inhales through the mouth piece, thereby resulting in a vacuum that causes a piston to move. In some embodiments, the moving piston is connected to a second piston that moves the formulation through a cannula and to the target site.

In another embodiment, the patient exhales or inhales through the nose with the device inserted to provide pneumatic pressure or vacuum to move the formulation. In some embodiments, when inserted, the device seals against the nostril with an elastomeric plug (ether by a face seal on the exterior of the nose or by a radial seal on the inner surface of the nostril). In some embodiments, when the patient sucks through the nose, this draws a vacuum in the nostril, this pulls the formulation out of the device, through a cannula, and to the target site. In some embodiments, the device seals against the nostril as described above, however the patient is instructed to blow, thereby building pressure in the nostril. In some embodiments, the device includes a port that allows air to flow from the nose and into the device, wherein the air flow pushes a plunger and forces the formulation out of the device, through a cannula, and to the target site. In some embodiments, the patient plugs the alternate nostril or the device may include a second elastomeric plug to block the second nostril.

In another embodiment, formulation may also wick into the target site by capillary force within the nasal cavity, such as within the narrow geometry of the olfactory region. This occurs when the formulation has appropriate surface tension and wetting angle with respect to the olfactory mucus. In some embodiments, having the wicking the formulation into target site is only viable in some patients due to naturally occurring variation in patient anatomy. In this embodiment, the formulation is contained within a bag that is connected to a cannula. In some embodiments, the cannula is placed in contact with the narrow top of the olfactory region. In some embodiments, the bag is partially depressed to fill the cannula with formulation and to have the formulation make contact with the narrow top of the olfactory region, wherein capillary pressure then draws the formulation out of the bag and into the olfactory region.

In another embodiment, formulation is fed into the nasal cavity by gravity. In some embodiments, a cannula is inserted into the patient with the tip contacting the target site within the nasal cavity olfactory region. In some embodiments, a container (e.g. IV bag) holding the formulation is connected to the cannula and held above the olfactory. Gravity then forces the formulation from the container, through the cannula, and into the olfactory. In another embodiment, the patient is positioned with the head upside down (e.g. patient lies back on a table and the head is tilted back) so that the olfactory holds the formulation without requiring capillary force. In another embodiment, the patient is positioned with the head upside down so the formulation and the cannula is inserted partially into the nose (not into the olfactory). Gravity then causes the formulation to flow out of the cannula, down the top of the nasal cavity, and into the olfactory.

In some embodiments, the formulation may be placed at the target site directly using a rigid body (e.g. spoon, swab). In some embodiments, the body may be articulated to improve placement. In some embodiments, the body is an endoscope or a cannula sheathing. In some embodiments, a swab saturated with the formulation is placed at the target site, left for a period of time, and then removed to recover the formulation.

### Capturing Biological Material

Method 100 further comprises allowing the delivered formulation to capture biological material at 108. As disclosed herein, the term “biological material” and related terms refers to material produced by a living organism, and includes cerebrospinal fluid (CSF), one or more microbes of a patient's microbiome, biomarkers, sub-combination of biomarkers, one or more pathogens, one or more components of a patient's metabolome, other components, or any combination thereof. In some embodiments, the one or more pathogens is a virus, or a portion or derivative of the virus. For example, in some embodiments, the virus is SARS CoV-2.

In some embodiments, once the formulation is delivered to the target site (for e.g., olfactory region), biological material, such as a biomarker of interest, located in the target site is absorbed by the formulation bolus and/or the formulation causes the biomarker of interest to diffuse directly into the formulation from the surrounding tissue and fluid. In some embodiments, the biological material adheres to the delivered formulation.

In some embodiments, during capture of the biological material by the formulation, air flow (from patient breathing or generated by the device) is used to evaporate fluid from the formulation bolus. In some embodiments, the patient provides a controlled breathing rate to ensure an appropriate level of evaporation. In some embodiments, the device includes a second cannula inserted into the nose and a spring driven bellows, electric fan, canister of compressed gas, or other source that will force air or gas over the bolus. This increases the bolus' osmolality and allows for additional fluid to be absorbed by the formulation from the target site, and concentrates the sample of relevant biological material in the formulation bolus.

In some embodiments, to prepare for capture of the biological material by the formulation, the patient is induced to a) increase mucus production, b) decrease mucus production, c) increase blood flow, d) decrease blood flow, or e) increase intracranial pressure, to improve the transport of biomarkers of interest to the formulation bolus when it is applied. In some embodiments, the patient is induced using medications.

In some embodiments, energy is applied to enhance the transport of nasal cavity fluid containing biological material, and/or to enhance the biological material transport, including biomarker transport, from adjacent tissue/fluid into the formulation. For example, in some embodiments, heat is applied to the formulation through UV/VIS/IR light, ohmic heating of the formulation or conduction from a heated element within the device. In some embodiments, electric/magnetic fields are applied to move biological material, including biomarkers of interest, into the formulation. In some embodiments, vibration, sound, or ultrasound energy is applied to agitate the formulation or patient to increase transport.

In some embodiments, the device may repeatedly eject and recover the formulation to enhance recovery of the biological material from a target site within the nasal cavity or the olfactory region. In some embodiments, the device may pulse a small portion of the bolus in and out to enhance recovery.

In some embodiments, the device produces a flow of formulation that leaves the device, washes over the target site, and is recovered in a series of boluses or in a continuous flow.

### Recovery of Formulation and Captured Biological Material

Method 100 further comprises withdrawing the formulation and captured biological material (i.e. collecting the captured biological material) from a target site within the nasal cavity, such as the olfactory region at 110. In some embodiments, the formulation and biological material are withdrawn using a cannula, or other microfluidic channel. In some embodiments, the formulation and biological material are withdrawn via the same orifice and cannula used for delivery of the formulation. In some embodiments, the formulation and biological material are withdrawn via a different orifice and cannula used for delivery of the formulation. In some embodiments, the formulation and biological material are captured within the same container used for containing the formulation prior to delivery to the target site. In some embodiments, the formulation and biological material are captured within a different container from the container used for containing the formulation prior to delivery to the target site.

In some embodiments, a pressure difference moves the formulation and biological material from the target site through a fluid path (e.g. a cannula or a microfluidic channel) and into the device. In some embodiments, the pressure difference is provided by spring force, motor force, pneumatic pressure, vacuum, or force provided by the hand, moving the plunger of a carpule (single, multi-chamber), syringe (single, multi-chamber), or pipette. In some embodiments, the pressure difference is provided by relaxation of a previously compressed single use pipette, blow-fill-seal container (e.g. MicroDose™ single use unit), bellows, microfluidic cartridge, or molded bag. In some embodiments, the pressure difference is provided by a pump (e.g. peristaltic pump, piston pump, gear pump, etc.). In some embodiments, vacuum is provided by an evacuated container (e.g. vacutainer, bottle, machined chamber), by relaxation of a previously compressed disposable pipette, bulb syringe, or bellows, or by a pump.

In some embodiments, the patient exhales or inhales, through the mouth, into the device to provide pneumatic pressure or vacuum to move the formulation, including the biological material, from the target site into the device. In some embodiments, the patient exhales or inhales through the nose with the device inserted to provide pneumatic pressure or vacuum to move the formulation, including the biological material, from the target site into the device. In some embodiments, the device includes a mouthpiece that rests in the patient's mouth when the device is inserted in the nose. In some embodiments, the patient inhales through the mouth piece. In some embodiments, the applied vacuum moves a piston which draws formulation out of the target site, through a cannula, and into the device. In some embodiments, the applied vacuum also draws the formulation into a bag or bellows. In some embodiments, the formulation may also be drawn into a fluid knockout chamber like a suction canister. In some embodiments, the patient blows on the mouth piece. In some embodiments, the applied pressure causes a piston to move. In some embodiments, the moving piston is connected to a second piston that draws fluid through a cannula and into the device.

In another embodiment, the patient exhales or inhales through the nose with the device inserted to provide pneumatic pressure or vacuum to move the formulation, including the biological material, into the device. In some embodiments, when inserted, the device seals against the nostril with an elastomeric plug (ether by a face seal on the exterior of the nose or by a radial seal on the inner surface of the nostril). In some embodiments, when the patient exhales through the nose, this creates pressure in the nasal cavity and pushes fluid out of the target site, through a cannula, and into the device. In some embodiments, the device seals against the nostril as described above, however the patient is instructed to inhale, thereby drawing a vacuum in the nose. In some embodiments, the device includes a port that allows air to flow from the device and into the nose. In some embodiments, inside the device, the air flow moves a plunger which draws fluid into the device through a cannula. In some embodiments, the patient plugs the alternate nostril or the device includes a second elastomeric plug to block the second nostril.

In some embodiments, the formulation, including the biological material, is wicked from the target site into the device through capillary pressure, drawing the fluid into an absorbent swab, absorbent pad, sponge, wick, or lateral flow assay strip. In some embodiments, a rigid body, such as a thin aluminum rod, with an absorbent pad attached to the tip is used to facilitate contact with the biological material and formulation at the target site. In some embodiments, the rigid body is inserted into the nose so that the absorbent pad contacts and absorbs the formulation, wherein the pad is then withdrawn from the nose. In some embodiments, the pad is compressed to push the formulation out into a standard sample holding container. In some embodiments, the entire absorbent pad is placed in a body of standard preservative inside a sample holding container. In some embodiments, the rod and pad are sheathed so that the pad is not contaminated during insertion.

In some embodiments, the wicking element provides a flow path from the sampling site out of the nose into the device. In some embodiments, the formulation, including the captured biological material, is wicked through a flow path (e.g. a cannula or microfluidic channel). In some embodiments, a cannula that is filled with absorbent, open cell foam is placed in the olfactory, wherein the tip of the cannula has an exposed section of foam. In some embodiments, when the foam contacts the formulation, the formulation is wicked into the foam and down the cannula. In some embodiments, at the base of the cannula, fluid is wicked into a chamber filled with absorbent foam. In some embodiments, the foam includes lyophilized preservatives to protect the captured biological material. In some embodiments, the foam chamber is replaced by a lateral flow assay strip to provide point of care diagnostics.

In some embodiments the formulation viscosity reduces after sampling is complete and the formulation, including the biological material, simply drains from the nose. For example, in some embodiments, the formulation includes chemical agents that react or decay a) after a time delay, b) with air, c) with a separately introduced gas or liquid, d) or with patient body fluids such that the formulation decreases in viscosity. In some embodiments, the formulation is thickened by long chain natural sugar polymers (polysaccharides). In some embodiments, the formulation, including the biological material, is mixed with enzymes in a dual chamber carpule before delivery. In some embodiments, the enzymes break down the sugar polymers over time which reduces the viscosity of the formulation. In some embodiments, the formulation then drains out of the nose by gravity and is captured in an appropriate container (e.g. bottle, jar, or lateral flow assay strip).

In some embodiments, the formulation, including the biological material, changes into a cohesive body and is pulled out of the nose with tensile force allowing preservation of the biological material according to its target site localization. For example, in some embodiments, the formulation includes solvents (e.g. ethanol) that evaporate to transform the formulation into a cohesive body. In some embodiments, the formulation includes chemical agents that a) react after a time delay, b) react with air, c) react with a separately introduced gas or liquid, or react with patient body fluids to form a cohesive body. In some embodiments, the cohesive body is then pulled from, blown from, or falls out of the nose to recover the formulation.

In some embodiments, the formulation only stays at the target site because of the patient's position. In some embodiments, a change in the patient's position allows the formulation to drain out of the nose.

In some embodiments, the captured biological material from a targeted sub-region is preserved with respect to its localization (geography).

### Analysis of Collected Biological Material

Method 100 further comprises analyzing the biological material at 112. For example, in some embodiments, biological material, such as biomarkers (e.g. proteins) captured by the formulation are detected or quantified to inform a diagnosis. In some embodiments, analysis is conducted 1) immediately with a point-of-care assay system (e.g. lateral flow assay) and/or 2) at a later time and/or at another site, wherein the formulation may be mixed with preservative.

In some embodiments, for point-of-care diagnostics, the formulation is removed from the device (e.g. drawn with a disposable pipette) and placed onto a separate point of care system (e.g. lateral flow assay strip). In some embodiments, the point of care system is integrated within the device. For example, in some embodiments, the formulation is recovered by capillary pressure provided by a lateral flow assay strip and the formulation is drawn from the target site directly in the lateral flow assay strip. In some embodiments, a recovery vessel for receiving the formulation and/or biological material comprises chemicals that produces a color change to indicate the presence of a specific biological material, such as a target biomarker (for example, SARS-CoV-2 pathogens), captured within the formulation.

In some embodiments, for diagnostics at another site, the formulation is mixed with a preservative solution and placed in an appropriate container for transport. In some embodiments, the formulation is removed from the device (e.g. drawn with a disposable pipette) and placed onto a separate container that contains preservative (e.g. a jar with lyophilized preservative). In some embodiments, the formulation is drawn into a transportable container (e.g., recovery vessel), containing preservative, that is integrated into the device (e.g. a carpule, or syringe) that can be removed from the device for transport (for example, a recovery vessel may contain preservative to stabilize the biological material). In some embodiments, the formulation is drawn into a section of the device that contains preservative that can be broken off or otherwise detached from the device for transport. In some embodiments, the formulation itself may also contain the required preservative elements.

In some embodiments, the formulation is configured to preserve and enable downstream processing of the collected biological material. In some embodiments, such downstream processing includes 16S sequencing, metagenomic sequencing, transcriptomics, mass spectroscopy and live bacterial culturing. In some embodiments, factors impacting the compositions, systems, methods, and apparatus disclosed herein is 1) the collection of an appropriate volume of biological material, and 2) appropriate localized preservation of materials from the site of collection through sample preparation steps. In some embodiments, given the geography of the nasal anatomy, precision localized sampling would be in the range of discrete millimeters.

### Example Apparatus and Methods

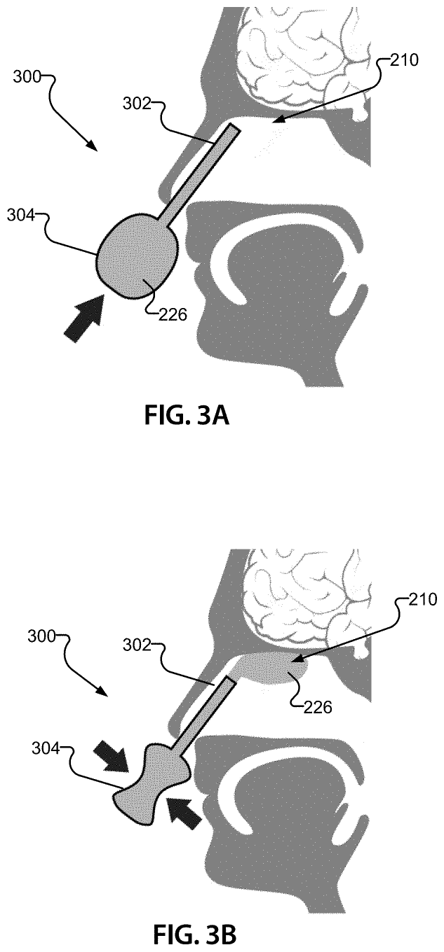

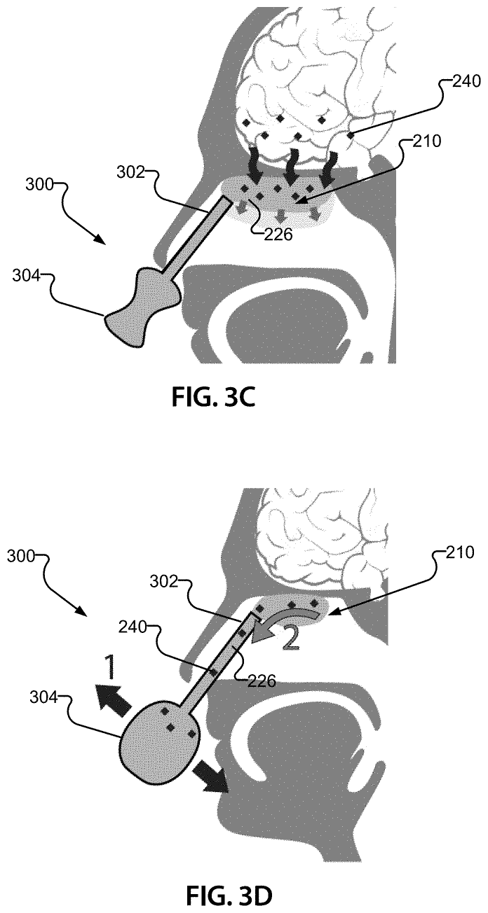

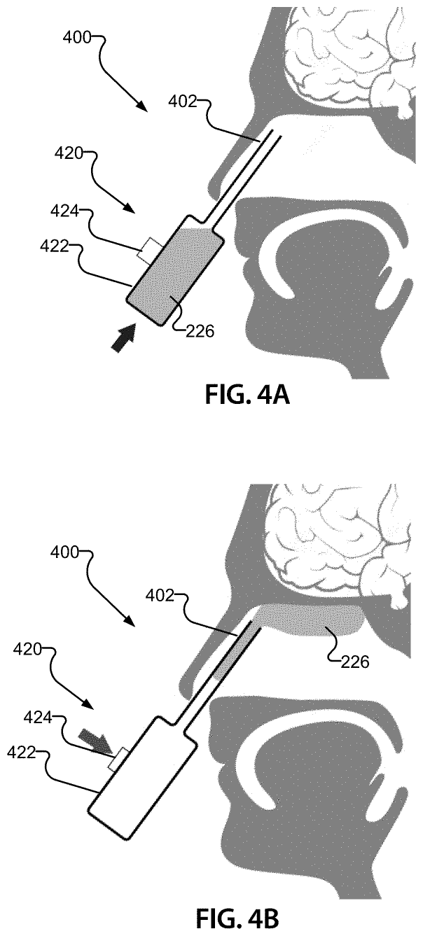

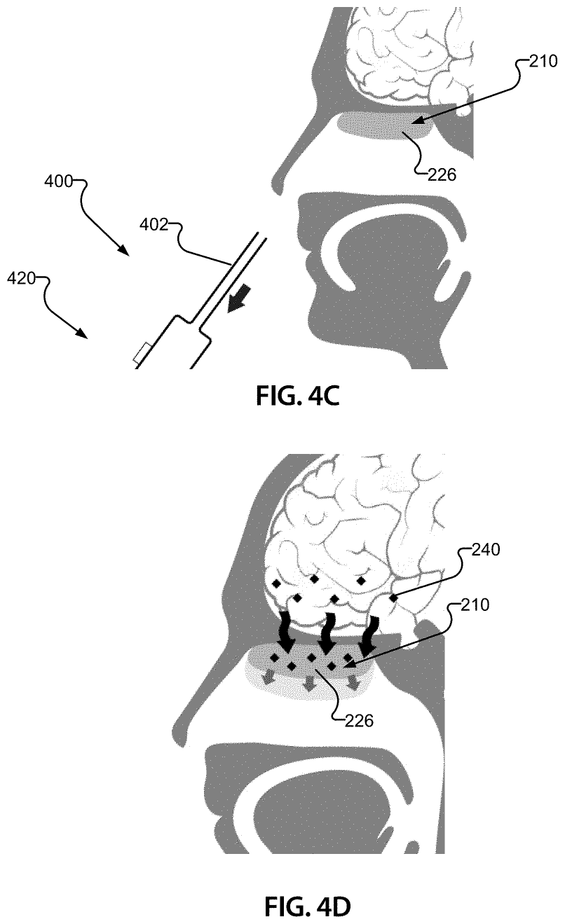

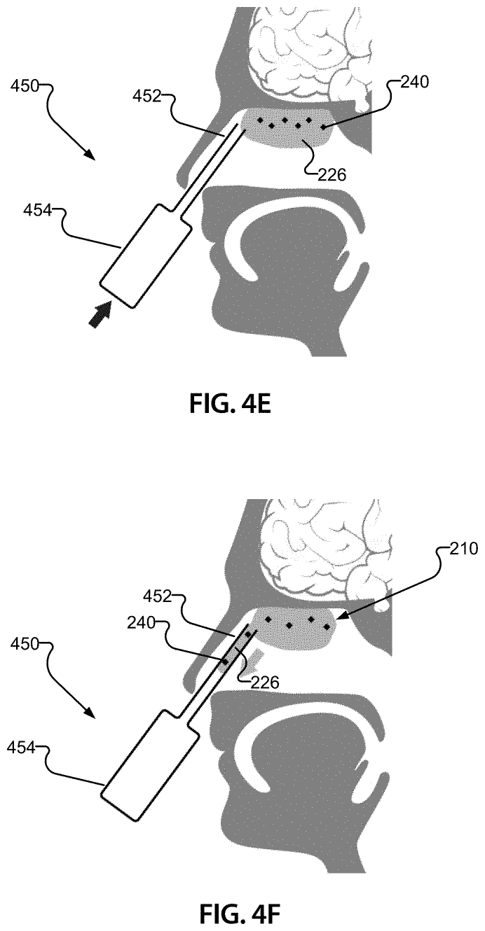

As described in more detail below, FIGS. 2A-2F, FIGS. 3A-3D, and FIGS. 4A-4F illustrate exemplary embodiments for collecting biological material from a nasal cavity of a patient, such as the olfactory region of the nasal cavity. The exemplary embodiments and methods as described herein are also applicable to collecting biological material from other regions of the nasal cavity, as disclosed herein. FIGS. 2A-2F show steps of an exemplary method using a device 200 comprising a common cannula 202 and separate containers 220 and 250 for delivering and recovering the formulation respectively. FIGS. 3A-3D show steps of an exemplary method using a device 300 comprising a cannula 302 and an attached bulb 304 for both delivering and recovering the formulation. FIGS. 4A-4F shows steps of an exemplary method using a device 400 comprising a container 420 with a cannula 402 for formulation delivery, and another container 450 with a cannula 452 for formulation recovery. Details of these example devices and their operation are described below.

As shown in FIG. 2A, the device 200 comprises a flexible, rigid, or conforming cannula 202. In some embodiments, the cannula at 202 incorporates a sheathing mechanism to protect the olfactory region from contamination from the lower nasal cavity and protect the biological material from contamination during the acquisition phase. In some embodiments, the device 200 comprises a container 220 (FIG. 2B) with a body for holding the formulation 226. In some embodiments, the container 220 comprises a deployment mechanism 224 for ejecting the formulation 226 from the container 220 as shown in FIG. 2B. In some embodiments, the container 220 is removably attached to the cannula 202. In some embodiments, the container 220 comprises a carpule 222.

In some embodiments, the device 200 is located against the external base of the nose. For example, in some embodiments, as shown in FIG. 2A, the device comprises a clip 212 mounted on a clip base 206. The clip 212 provides an anatomical reference point for accurate placement of the cannula 202 and maintains consistent placement of the cannula 202.

In some embodiments, the cannula 202 is a fixed length suited for the general population. In some embodiments, the cannula 202 is of a variable length that is set to a patient's specific measurements. For example, in some embodiments, the cannula 202 is slidably attached to the base 206 such that it may be moved relative to the clip 212. In some embodiments, the cannula 202 includes graduations (e.g. markings on the cannula) to assist placement. The graduations may be used to insert the cannula to a pre-determined depth, for example, such that the tip of the cannula 202 reaches the olfactory region 210 but does not damage the tissue. In some embodiments, the pre-determined depth is, for example, determined by taking a pre-insertion measurement with a CT scan or otoscope for an individual patient or by utilizing a maximum safe length as determined by analyzing a database of pool anthropometric measurements of the nasal cavity.

In some embodiments, the deployment mechanism comprises any delivery mechanism as disclosed herein. For example, as shown in FIG. 2B, the deployment mechanism 224 is coupled to the container 220, and is configured such that when the deployment mechanism 224 is activated, the formulation 226 is ejected from the container 220, through the cannula 202 and is deposited in the olfactory region 210 of the patient, as shown in FIG. 2C. In some embodiments, the deployment mechanism is activated by a user. In some embodiments, the cannula 202 comprises an orifice that is positioned to deliver the formulation 226 to a targeted sub-region (not shown).

In some embodiments, the deployment mechanism 224 comprises a button, a spring, and a plunger (not shown). When the button is pressed, this releases the spring, which moves the plunger to force the formulation 226 from the container 220 into the olfactory region 210. The deployment mechanism 224 may take other forms in other embodiments.

As described above, in some embodiments the formulation has a higher osmolality than the mucus in the olfactory region 210. In some embodiments, the formulation includes sugars to both increase osmolality and to create a cohesive fluid body that can be fully extracted. In some embodiments, the increased osmolality creates an osmotic pressure gradient that favors uptake of the biological material, such as a specific biomarker target 240, into the formulation 226 deposited in the olfactory region 210, as shown in FIG. 2D. In some embodiments the formulation 226 is shear thinning to promote distribution of the formulation 226 into the narrow spaces within the olfactory region 210.

In some embodiments, recovery of the formulation and/or biological material comprises any recovery mechanism as disclosed herein. For example, as shown in FIG. 2E, in some embodiments, the container 220 is disconnected from the cannula 202 and replaced with a recovery vessel 250. In some embodiments, the recovery vessel 250 comprises a body configured to hold the withdrawn formulation and/or biological material. In some embodiments, the recovery vessel 250 comprises a carpule 252, a deployment button 254, a spring (not shown) and/or a plunger (not shown). As shown in FIG. 2F, when the deployment button 254 is pressed, the spring moves the plunger which draws the formulation 226 and biological material 240 through the cannula 202 into the recovery vessel 250. In some embodiments, system geometry and rate of travel of the plunger are controlled (e.g. by dampening) to ensure the formulation 226 is not drawn in too quickly (e.g. minimize or prevent shear forces experienced by the captured biological material 240 from affecting analysis results through damage to the contents of the captured biological material, and minimizing or preventing air from being recovered instead of the full amount of formulation and biological material).

The formulation 226 is any formulation as disclosed herein. For example, in some embodiments, the formulation shown in FIG. 2F is crosslinked upon exposure to air or other means (as described herein), thereby changing the formulation into a semi-solid state. In some embodiments, the semi-solid formulation facilitates preservation of the captured biological material according to its target site localization while the semi-solid formulation is withdrawn and preserved in the vessel at 252.

In some embodiments, the cannula 202 is sheathed to prevent or minimize cross contamination of the biological material and/or contamination of the olfactory region through cannula inoculation from the lower nasal anatomy.

FIG. 3A shows another embodiment of a device 300 comprising a cannula 302 and a flexible bulb 304. As shown in FIG. 3B, pressing on the flexible bulb 304 pushes the formulation 226 through the cannula 302 and into the olfactory region 210. In some embodiments, the cannula 302 comprises an orifice that is positioned to deliver the formulation 226 to a targeted sub-region (not shown). The formulation is any formulation as disclosed herein.

FIG. 3C shows a formulation 226 with a higher osmolality than the biological material 240, creating an osmotic pressure gradient that favors uptake of the biological material, including biomarkers of interest contained therein 240, into the formulation 226.

As shown in FIG. 3D, the flexible bulb 302 is allowed to relax, drawing the formulation 226 and biological material 240 through the cannula 302 back into the bulb 304.

In some embodiments, the cannula 302 is sheathed to prevent or minimize cross contamination of the biological material and/or contamination of the olfactory region through cannula inoculation from the lower nasal anatomy.

FIGS. 4A-D shows a device 400 comprising a container 420 holding formulation 226 with a deployment mechanism 424 configured to eject the formulation through a cannula 402, similar to the operation of device 200 described above. In some embodiments, the container 420 comprises a carpule 422. In some embodiments, the cannula 402 comprises an orifice that is positioned to deliver the formulation 226 to a targeted sub-region (not shown). In some embodiments, the deployment mechanism comprises any delivery mechanism as disclosed herein. The formulation is any formulation as disclosed herein.

As shown in FIG. 4E, a recovery device 450 may be inserted into the patient's nose. In some embodiments, the recovery device comprises a body for holding the formulation and/or biological material after being withdrawn. In some embodiments, the recovery device comprises a recovery cannula different from the cannula used to deliver the formulation to the olfactory region. In some embodiments, the recovery cannula is detachably coupled to a recovery vessel. In some embodiments, the recovery device comprises any recovery mechanism as disclosed herein. For example, in some embodiments, the recovery device has a chamber 454 filled with wicking material and a recovery cannula 452 filled with wicking material configured to wick fluid. In some embodiments, the wicking material comprises natural or synthetic fibers, spun, woven or randomly oriented, and/or capillary tubes that wick fluid. In some embodiments, the recovery device 450 and wicking material is used to draw the formulation 226 and biological material 240 from the patient's olfactory region 210, as shown in FIG. 4F.

In some embodiments, the cannula 402 is sheathed to prevent or minimize cross contamination of the biological material and/or contamination of the olfactory region through cannula inoculation from the lower nasal anatomy.

The foregoing discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

### Example for Housekeeper Protein Detection in Human Sample Fluids

Cerebrospinal fluid (CSF)-predominant proteins Tau and Human Prostaglandin-H2 D-isomerase (PTGDS) have been successfully detected in human fluids. Commercial sandwich ELISA kits and qualitative mass spectrometry (LC-MS/MS) analysis were used to assay human CSF, nasal fluid (NF) and nasal lavage (NL). Mass spectrometry detected PTGDS in both CSF and NF, but PTGDS was likely too dilute to be detected in NL. The ELISA kit utilized was not able to detect PTGDS in CSF, thus invalidating its further use. Tau proteins were detected by ELISA at expected levels in CSF. Mass spectrometry was unable to detect Tau—likely due to its naturally low abundance. Tau exists at approximately 1000-fold lower concentration than PTGDS in CSF and may fall below the detection limit for this mass spec protocol. The ELISAs detected excessively high levels of both Tau and PTGDS from NF, and a lesser level of PTGDS in NL. This data was not corroborated by spectrometry analysis, and is likely due to non-specific reactions between the sticky nasal material and the ELISA kits' detection system. Table 3 and 4 provide results for the detection analysis for Tau and PTGDS, wherein the first four samples listed were diluted in ELISA sample diluent, and the last listed sample (Pooled Human Cerebral Spinal Fluid) was diluted in a synthetic CSF analogue fluid.

TABLE 3
Results for Human Tau Protein Detected using ELISA

| | | | | | | Conc. Tau cited | | |
|---|---|---|---|---|---|---|---|---|
| | Dilution | Expected | Measured | | | in Literature | | Total |
| | Factor | Tau Conc. | Tau Conc. | | | (Normal Sample) | | Protein |
| Sample Description | Reported | (pg/mL) | (pg/mL) | Stdev | CV % | (pg/mL) | pH | (mg/mL) |
| Pooled Human Cerebral Spinal Fluid | Neat | N/A | 613.75 | 97.94 | 16.00 | [symbol] 100 to 700 | TBD | TBD |
| Pooled Human Nasal Fluid | 4 | N/A | 1978.14 | 73.60 | 3.70 | <87 | TBD | TBD |
| Single Donor Human Nasal Lavage | Neat | N/A | <15.6 | 19.25 | 74.00 | No info | 7.6 | 0.116 |
| Control Tau Protein | N/A | 2000000 | <15.6 | 0.00 | 0.00 | N/A | N/A | N/A |
| Pooled Human Cerebral Spinal Fluid | 2 | N/A | 619.10 | 178.87 | 28.90 | [symbol] 100 to 700 [symbol] | N/A | N/A |

ELISA sensitivity: 15.6 pg/mL [symbol]
indicates data missing or illegible when filed

TABLE 4
Results for Human PTDGS Protein Detected using ELISA

| | | Expected | Measured | | | Conc. PTGDS cited | | |
|---|---|---|---|---|---|---|---|---|
| | Dilution | PTGDS | PTGDS | | | in Literature | | Total |
| | Factor | Conc. | Conc. | | | (Normal Sample) | | Protein |
| Sample Discription | Reported | (ng/mL) | (ng/mL) | Stdev | CV % | (ng/mL) | pH | (mg/mL) |
| Pooled Human Cerebral Spinal Fluid | 1 | N/A | <0.78 | N/A | N/A | 16,600 to 20,000 | 8.8 | 0.867 |
| Pooled Human Nasal Fluid | 64 | N/A | >3200 | N/A | N/A | 50 to 1200 | 7.7 | 9.666 |
| | | | | | | <3 to 120 | | |
| Single Donor Human Nasal Lavage | 1 | N/A | 17.06 | 0.29 | 1.70 | No info | 7.6 | 0.116 |
| Control PTGDS Protein | N/A | 1600 | <0.78 | N/A | N/A | N/A | N/A | N/A |
| Pooled Human Cerebral Spinal Fluid | 1 | N/A | <7.8 | N/A | N/A | 16,600 to 20,000 | 8.8 | 0.867 |

ELISA sensitivity: 0.78 ng/mL

LC/MS/MS analysis of the samples was able to detect an additional 147 proteins in CSF not found in either NF or NL. These proteins offer a choice of potential markers for tracking CSF fluid infiltration in patients experiencing rhinorrhea. Additionally, numerous CSF-occurring proteins were detected in either NF or NL. Using a quantitative approach, normal baseline values can be established for choice proteins, and diseases of the central nervous system that are attributed to increased protein levels can be diagnosed. Finally, mass spectrometry analysis detected numerous proteins in NL that did not occur NF (and vis versa). This indicates that a targeted sampling approach can be highly effective to capture proteins in geometrically disparate regions of the olfactory system. Tailoring the sampling method can offer rapid detection of and early diagnosis of site-specific diseases.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

What is claimed is:

1. A method of collecting biological material from an olfactory region of a patient, comprising:
a. providing a formulation configured to capture the biological material;
b. inserting a delivery device comprising a delivery orifice into a nasal cavity of the patient;
c. delivering the formulation via the delivery device into the olfactory region or a targeted sub-region of the olfactory region of the patient;
d. allowing the formulation to capture the biological material; and,
e. withdrawing at least a portion of the formulation and the biological material captured therein, thereby collecting the biological material.

2. The method of claim 1, wherein the delivery orifice is positioned such that the delivery of the formulation is to the targeted sub-region of the olfactory region.

3. The method of claim 1 or 2, further comprising preserving the composition of the formulation and/or captured biological material when being withdrawn.

4. The method of any one of claims 1-3, wherein the biological material comprises cerebrospinal fluid, one or more microbes of the patient's microbiome, one or more components of the patient's metabolome, one or more pathogens, and/or one or more biomarkers of interest.

5. The method of claim 4, wherein the one or more pathogens comprise a virus or a portion or derivative thereof.

6. The method of claim 5, wherein the virus is SARS CoV-2.

7. The method of any one of claims 1-6, wherein the delivery device comprises a cannula and/or microfluidic channel, and wherein inserting the delivery device comprises inserting the cannula and/or microfluidic channel into the nasal cavity of the patient.

8. The method of claim 7, further comprising determining a length of the patient's nasal cavity from the nostril to the olfactory region and inserting the cannula and/or microfluidic channel to a pre-determined depth based on the determined length.

9. The method of any one of claims 1-8, further comprising placing a reference device on the face of the patient so as to provide an anatomical reference point for accurate placement of the delivery orifice into the nasal cavity.

10. The method of any one of claims 1-9, wherein the biological material is captured from the targeted sub-region of the olfactory region.

11. The method of any one of claims 1-10, wherein the delivery device comprises a sheath configured to minimize or prevent contamination of the cannula and/or microfluidic channel, the delivery orifice, the formulation and/or the captured biological material from non-olfactory regions of the nasal cavity, and/or from regions of the olfactory region other than the targeted sub-region of the olfactory region.

12. The method of claim 11, wherein the sheath comprises a protective coating disposed about the cannula and/or microfluidic channel.

13. The method of claim 11, wherein the sheath comprises a cover or sleeve disposed about the cannula and/or microfluidic channel.

14. The method of any one of claims 1-13, further comprising inducing the patient so as to increase mucous production or decrease mucous production to facilitate the capture and/or collection of the biological material.

15. The method of any one of claims 1-14, further comprising inducing the patient so as to increase blood flow or decrease blood flow to facilitate the capture and/or collection of the biological material.

16. The method of any one of claims 1-15, further comprising inducing the patient so as to increase intracranial pressure to facilitate the capture and/or collection of the biological material.

17. The method of any one of claims 1-16, further comprising applying energy to facilitate the capture and/or collection of the biological material.

18. The method of claim 17, wherein applying energy comprises applying heat to the formulation through UV/VIS/IR light, through ohmic heating of the formulation, or through conduction from a heated element within the delivery device.

19. The method of any one of claims 1-18, wherein electric and/or magnetic fields are applied to facilitate the capture of the biological material by the formulation.

20. The method of any one of claims 1-19, wherein the delivery device is configured to deliver a flow of formulation to the olfactory region or targeted sub-region of the olfactory region, such that the flow of formulation is withdrawn as a continuous flow.

21. The method of any one of claims 1-20, further comprising repeating the method of any one of claims 1-20 so as to increase the collection of the biological material.

22. The method of any one of claims 1-21, wherein the formulation is the formulation of any one of claims 108-174.

23. A method of collecting biological material from a nasal cavity of a patient, comprising:
a. providing a formulation configured to capture the biological material;
b. inserting a delivery device comprising a delivery orifice into the nasal cavity or a targeted subregion of the nasal cavity of the patient;
c. delivering the formulation via the delivery device into the nasal cavity of the patient;
d. allowing the delivered formulation to capture the biological material; and,
e. withdrawing at least a portion of the formulation and the biological material captured therein.

24. The method of claim 23, wherein the delivery orifice is positioned such that the delivery of the formulation is to the targeted sub-region of the nasal cavity.

25. The method of claim 23 or 24, further comprising preserving the composition of the formulation and/or captured biological material when being withdrawn.

26. The method of any one of claims 23-25, wherein the biological material comprises cerebrospinal fluid (CSF), one or more microbes of the patient's microbiome, one or more components of the patient's metabolome, one or more pathogens, and/or one or more biomarkers of interest.

27. The method of claim 26, wherein the one or more pathogens comprise a virus or a portion or derivative thereof.

28. The method of claim 27, wherein the virus is SARS CoV-2.

29. The method of any one of claims 23-28, wherein the delivery device comprises a cannula and/or microfluidic channel, and wherein inserting the delivery device comprises inserting the cannula and/or microfluidic channel into the nasal cavity of the patient.

30. The method of claim 29, further comprising determining a length of the patient's nasal cavity from the nostril to the olfactory region and inserting the cannula and/or microfluidic channel to a pre-determined depth based on the determined length.

31. The method of any one of claims 23-30, further comprising placing a reference device on the face of the patient so as to provide an anatomical reference point for accurate placement of the delivery orifice into the nasal cavity.

32. The method of any one of claims 23-31, wherein the biological material is captured from a targeted region of the nasal cavity.

33. The method of claim 32, wherein the delivery device comprises a sheath configured to minimize or prevent contamination of the cannula and/or microfluidic channel, the delivery orifice, the formulation and/or the captured biological material from a non-targeted region of the nasal cavity.

34. The method of claim 33, wherein the sheath comprises a protective coating disposed about the cannula and/or microfluidic channel.

35. The method of claim 33, wherein the sheath comprises a cover or sleeve disposed about the cannula and/or microfluidic channel.

36. The method of any one of claims 23-35, further comprising inducing the patient so as to increase mucous production or decrease mucous production to facilitate the capture and/or collection of the biological material.

37. The method of any one of claims 23-36, further comprising inducing the patient so as to increase blood flow or decrease blood flow to facilitate the capture and/or collection of the biological material.

38. The method of any one of claims 23-37, further comprising inducing the patient so as to increase intracranial pressure to facilitate the capture and/or collection of the biological material.

39. The method of any one of claims 23-38, further comprising applying energy to facilitate the capture and/or collection of the biological material.

40. The method of claim 39, wherein applying energy comprises applying heat to the formulation through UV/VIS/IR light, through ohmic heating of the formulation, or through conduction from a heated element within the delivery device.

41. The method of any one of claims 23-40, wherein electric and/or magnetic fields are applied to facilitate the capture of the biological material by the formulation.

42. The method of any one of claims 23-41, wherein the delivery device is configured to deliver a flow of the formulation to the nasal cavity or targeted sub-region of the nasal cavity, such that the flow of formulation is withdrawn as a continuous flow.

43. The method of any one of claims 23-42, further comprising repeating the method of any one of claims 23-42 so as to increase the collection of the biological material.

44. The method of any one of claims 23-43, wherein the formulation is the formulation of any one of claims 108-174.

45. An apparatus for collecting biological material from a nasal cavity of a patient, comprising:
a. a first body containing a formulation configured to capture the biological material;
b. a first cannula and/or a microfluidic channel comprising a delivery orifice configured for positioning in the nasal cavity of the patient and fluidly connected to the first body;
c. a deployment mechanism for delivering the formulation through the first cannula and/or microfluidic channel into the nasal cavity of the patient, so as to capture biological material from the nasal cavity of the patient; and
d. a collection device for collecting the captured biological material from the nasal cavity of the patient.

46. The apparatus of claim 45, wherein the delivery orifice is configured such that the delivery of the formulation is to the targeted sub-region of the nasal cavity.

47. The apparatus of claim 45, wherein the delivery orifice is configured such that the delivery of the formulation is to an olfactory region of the nasal cavity.

48. The apparatus of claim 47, wherein the delivery orifice is configured such that the delivery of the formulation is to a targeted sub-region of the olfactory region.

49. The apparatus of any one of claims 45-48, wherein the biological material comprises cerebrospinal fluid (CSF), one or more microbes of the patient's microbiome, one or more components of the patient's metabolome, one or more pathogens, and/or one or more biomarkers of interest.

50. The apparatus of claim 49, wherein the one or more pathogens comprise a virus or a portion or derivative thereof.

51. The apparatus of claim 50, wherein the virus is SARS CoV-2.

52. The apparatus of any one of claims 45-51, wherein the biological material is captured from the targeted sub-region of the nasal cavity.

53. The apparatus of any one of claims 45-51, wherein the biological material is captured from an olfactory region of the nasal cavity.

54. The apparatus of claim 53, wherein the biological material is captured from a targeted sub-region of the olfactory region of the nasal cavity.

55. The apparatus of any one of claims 45-54, wherein the apparatus comprises a sheath configured to minimize or prevent contamination of the first body, the first cannula and/or microfluidic channel, the delivery orifice, the collection device, the formulation and/or the captured biological material from non-targeted regions of the nasal cavity.

56. The apparatus of claim 45-55, wherein the sheath is configured to minimize or prevent contamination of the first body, the first cannula and/or microfluidic channel, the delivery orifice, the collection device, the formulation and/or the captured biological material from non-targeted sub-regions of the olfactory region.

57. The apparatus claim 55 or 56, wherein the sheath comprises a protective coating disposed about the first cannula and/or microfluidic channel.

58. The apparatus of claim 55 or 56, wherein the sheath comprises a cover or sleeve disposed about the first cannula and/or microfluidic channel.

59. The apparatus of any one of claims 45-58, wherein the first body comprises a first container detachably coupled to the first cannula and/or microfluidic channel.

60. The apparatus of any one of claims 45-59, wherein the collection device comprises a second body detachably coupled to the first cannula and/or microfluidic channel.

61. The apparatus of any one of claims 45-60, wherein the collection device comprises a second body and a second cannula coupled to the second body.

62. The apparatus of any one of claims 45-61, wherein the collection device is configured to preserve the integrity and biological material according to its localization as captured from the nasal cavity.

63. The apparatus of any one of claims 45-62, wherein the deployment mechanism comprises a first actuator coupled to a first spring that is coupled to a first plunger.

64. The apparatus of any one of claims 45-63, wherein the apparatus further comprises a clip configured to couple with the patient's nose so as to facilitate the positioning of the delivery orifice.

65. The apparatus of claim 64, wherein the first cannula and/or microfluidic channel is configured to move relative to the clip.

66. The apparatus of any one of claims 45-65, wherein the collection device comprises a second actuator coupled to a second spring that is coupled to a second plunger.

67. The apparatus of any one of claims 45-66, wherein the first body comprises a carpule.

68. The apparatus of any one of claims 45-67, wherein the first cannula and/or microfluidic channel is a flexible cannula.

69. The apparatus of any one of claims 45-67, wherein the first cannula and/or microfluidic channel is a telescoping cannula.

70. The apparatus of any one of claims 45-69, wherein the apparatus comprises mechanical features to prevent hazardous forces being transmitted through the first cannula and/or microfluidic channel.

71. The apparatus of claim 70, wherein the mechanical features comprise a force limiting spring, a radial slip clutch, and/or an axial slip clutch.

72. The apparatus of any one of claims 45-71, wherein the targeted sub-region of the olfactory region is localized to regions of discrete millimeters within the olfactory region.

73. The apparatus of any one of claims 45-72, wherein the formulation is the formulation of any one of claims 108-174.

74. A system for collecting biological material from a nasal cavity of a patient, comprising:
a. a first body configured to contain a formulation;
b. a first cannula and/or a microfluidic channel comprising a delivery orifice configured for positioning in the nasal cavity of the patient and fluidly connected to the first body;
c. a deployment mechanism for delivering the formulation through the first cannula and/or microfluidic channel into the nasal cavity of the patient;
d. a collection device for collecting the biological material from the nasal cavity of the patient; and
e. the formulation, wherein the formulation is configured to capture the biological material.

75. The system of claim 74, wherein the delivery orifice is configured such that the delivery of the formulation is to a targeted sub-region of the nasal cavity.

76. The system of claim 74, wherein the delivery orifice is configured such that the delivery of the formulation is to an olfactory region of the nasal cavity.

77. The system of claim 76, wherein the delivery orifice is configured such that the delivery of the formulation is to a targeted sub-region of the olfactory region.

78. The system of any one of claims 74-77, wherein the biological material comprises cerebrospinal fluid (CSF), one or more microbes of the patient's microbiome, one or more components of the patient's metabolome, one or more pathogens, and/or one or more biomarkers of interest.

79. The system of claim 78, wherein the one or more pathogens comprise a virus or a portion or derivative thereof.

80. The system of claim 79, wherein the virus is SARS CoV-2.

81. The system of any one of claims 74-80, wherein the biological material is captured from a targeted region of the nasal cavity.

82. The system of any one of claims 74-80, wherein the biological material is captured from an olfactory region of the nasal cavity.

83. The system of claim 82, wherein the biological material is captured from a targeted sub-region of the olfactory region of the nasal cavity.

84. The system of any one of claims 74-83, wherein the system comprises a sheath configured to minimize or prevent contamination of the first body, the first cannula and/or microfluidic channel, the delivery orifice, the collection device, the formulation and/or the captured biological material from non-targeted regions of the nasal cavity.

85. The system of any one of claims 74-83, wherein the sheath is configured to minimize or prevent contamination of the first body, the first cannula and/or microfluidic channel, the delivery orifice, the collection device, the formulation and/or the captured biological material from non-olfactory regions of the nasal cavity or non-targeted sub-regions of the olfactory region.

86. The system of claim 84 or 85, wherein the sheath comprises a protective coating disposed about the first cannula and/or microfluidic channel.

87. The system of claim 84 or 85, wherein the sheath comprises a cover or sleeve disposed about the first cannula and/or microfluidic channel.

88. The system of any one of claims 74-87, wherein the first body comprises a first container detachably coupled to the first cannula and/or microfluidic channel.

89. The system of any one of claims 74-88, wherein the collection device comprises a second body detachably coupled to the first cannula and/or microfluidic channel.

90. The system of any one of claims 74-89, wherein the collection device comprises a second body and a second cannula coupled to the second body.

91. The system of any one of claims 74-90, wherein the collection device is configured to preserve the integrity and biological material according to its localization as captured from the nasal cavity.

92. The system of any one of claims 74-91, wherein the deployment mechanism comprises a first actuator coupled to a first spring that is coupled to a first plunger.

93. The system of any one of claims 74-92, wherein the apparatus further comprises a clip configured to couple with the patient's nose so as to facilitate the positioning of the delivery orifice.

94. The system of claim 93, wherein the first cannula and/or microfluidic channel is configured to move relative to the clip.

95. The system of any one of claims 74-94, wherein the collection device comprises a second actuator coupled to a second spring that is coupled to a second plunger.

96. The system of any one of claims 74-95, wherein the first body comprises a carpule.

97. The system of any one of claims 74-96, wherein the first cannula and/or microfluidic channel is a flexible cannula.

98. The system of any one of claims 74-96, wherein the first cannula and/or microfluidic channel is a telescoping cannula.

99. The system of any one of claims 74-98, wherein the system comprises mechanical features to prevent hazardous forces being transmitted through the cannula.

100. The system of claim 99, wherein the mechanical features comprise a force limiting spring, a radial slip clutch, and/or an axial slip clutch.

101. The system of any one of claims 74-100, wherein the targeted sub-region of the olfactory region is localized to regions of discrete millimeters within the olfactory region.

102. The system of any one of claims 74-101, wherein the formulation is of any one of claims 108-174.

103. A method of making a diagnosis of a patient, comprising:
a. performing the method of any one of claims 1-44, thereby collecting the biological material from the patient;
b. analyzing the collected biological material; and
c. based on the analysis of step b., making the diagnosis.

104. The method of claim 103, wherein analyzing the biological material comprises identifying and/or quantifying biomarkers, pathogens, and/or microbes in the collected biological material.

105. The method of claim 104, further comprising correlating the identified and/or quantified biomarkers, pathogens, and/or microbes with a corresponding physiological characteristic and/or medical condition.

106. The method of any one of claims 103-105, wherein analyzing the biological material comprises using a point-of-care assay system.

107. The method of claim 100, wherein the point-of-care assay system is configured to receive a sample of the collected biological material from the delivery device from any one of claims 1-44, the apparatus from any one of claims 45-73, or the system from any one of claims 74-102.

108. A formulation for collecting biological material from a nasal cavity of a patient, wherein the formulation is configured to capture biological material once delivered within the nasal cavity, the delivered formulation configured to be withdrawn from the nasal cavity with the biological material.

109. The formulation of claim 108, wherein the formulation is delivered to the olfactory region of the nasal cavity.

110. The formulation of claim 108 or 109, wherein the formulation is configured to capture biological material from a targeted sub-region of the olfactory region.

111. The formulation of any one of claims 108-110, wherein the delivered formulation is configured to preserve the captured biological material when being withdrawn.

112. The formulation of any one of claims 108-111, wherein the biological material comprises cerebrospinal fluid (CSF), one or more microbes of the patient's microbiome, one or more components of the patient's metabolome, one or more pathogens, and/or one or more biomarkers of interest.

113. The formulation of claim 112, wherein the formulation is configured to capture specific biological material.

114. The formulation of any one of claims 108-113, wherein the formulation comprises a buffered saline solution.

115. The formulation of claim 114, wherein the buffered saline solution is 100 mM phosphate buffered saline.

116. The formulation of any one of claims 108-115, wherein the formulation comprises one or more gelling agents and/or thickeners.

117. The formulation of any one of claims 108-116, wherein the formulation comprises a viscosity modifier to provide a desired viscosity for the formulation.

118. The formulation of claim 117, wherein the viscosity modifier comprises at least one of glycerol, pectin, and polyethylene glycol.

119. The formulation of claim 117 or 118, wherein the viscosity modifier comprises 25-75% of the formulation by volume.

120. The formulation of any one of claims 108-119, wherein the formulation has a higher osmolality than fluid in the nasal cavity, the olfactory region, or a targeted sub-region of the olfactory region of the patient.

121. The formulation of any one of claims 108-119, wherein the formulation has an osmolality equal to or less than fluid in the nasal cavity, the olfactory region, or a targeted sub-region of the olfactory region of the patient.

122. The formulation of any one of claims 108-121, wherein a desired osmolality of the formulation is achieved through inclusion of salts, sugars, starches, albumin, dextran, or combinations thereof in the formulation.

123. The formulation of any one of claims 108-122, wherein the delivered formulation has an osmolality adjusted such that said osmolality is equal to a targeted osmolality after a target volume of fluid other than the formulation has been withdrawn from the nasal cavity, olfactory region or the targeted sub-region of the olfactory region.

124. The formulation of any one of claims 108-123, wherein the osmolality of the formulation is configured to vary over time, so as to capture biological material from the nasal cavity, the olfactory region, or the targeted sub-region of the olfactory region at a desired rate.

125. The formulation of claim 124, wherein the osmolality of the formulation is configured to vary over time by inclusion of osmotic modifying agents in the formulation.

126. The formulation of claim 125, wherein the osmotic modifying agents comprise micro-encapsulated particles of one or more osmotic modifying agents.

127. The formulation of claim 125 or 126, wherein the one or more osmotic modifying agents comprise sodium chloride.

128. The formulation of any one of claims 125-127, wherein the micro-encapsulated particles comprise an enteric coating containing the one or more osmotic modifying agents.

129. The formulation of claim 128, wherein the enteric coating is configured to release the one or more osmotic modifying agents upon exposure to defined conditions for a defined time period.

130. The formulation of claim 129, wherein the defined conditions comprise one or more conditions selected from the group consisting of a temperature range, a pH range, and a defined shear force.

131. The formulation of any one of claims 108-130, wherein the formulation comprises an agent that promotes mucus production within the nasal cavity, the olfactory region, or the targeted sub-region of the olfactory region, so as to facilitate the capture of the biological material.

132. The formulation of claim 131, wherein the agent that promotes mucus production is capsaicin.

133. The formulation of any one of claims 108-132, wherein the formulation comprises one or more agents that thicken mucus within the nasal cavity, the olfactory region, or the targeted sub-region of the olfactory region, so as to prevent the delivered formulation from moving, thereby increasing residence time of the delivered formulation within the nasal cavity, the olfactory region or the targeted sub-region of the olfactory region.

134. The formulation of any one of claims 108-133, wherein the formulation is configured to change from a liquid state to a semi-solid state upon delivery to the nasal cavity, the olfactory region, or to the targeted sub-region of the olfactory region.

135. The formulation of claim 134, wherein the formulation is configured to initiate a cross-linking reaction upon delivery to the nasal cavity, the olfactory region, or to the targeted sub-region of the olfactory region.

136. The formulation of claim 135, wherein the formulation comprises two or more reagents.

137. The formulation of claim 136, wherein the two or more reagents are configured to mix upon delivery to the nasal cavity, the olfactory region, or to the targeted sub-region of the olfactory region, so as to initiate the cross-linking reaction, thereby changing the formulation into a semi-solid state.

138. The formulation of any one of claims 134-137, wherein the formulation comprises a non-Newtonian fluid.

139. The formulation of claim 138, wherein the formulation changes from a liquid state to a semi-solid state at a temperature of about that of human body temperature.

140. The formulation of claim 138, wherein the formulation changes from a liquid state to a semi-solid state at a temperature of about 35° C. to about 40° C.

141. The formulation of claim 138, wherein the formulation changes from a liquid state to a semi-solid state at a temperature of about 37° C.

142. The formulation of any one of claims 134-141, wherein the formulation comprises a Bingham plastic.

143. The formulation of claim 142, wherein the formulation behaves as a liquid when subject to shear force during delivery to the nasal cavity, the olfactory region, or to the targeted sub-region of the olfactory region.

144. The formulation of claim 143, wherein the formulation behaves as a semi-solid when not subject to shear force.

145. The formulation of any one of claims 134-144, further comprising a tail formed through the delivery and partial solidification of the formulation.

146. The formulation of claim 145, wherein the tail is configured to be mechanically removed, thereby facilitating removal of the captured biological material.

147. The formulation of any one of claims 134-146, wherein the semi-solid state of the formulation is configured to preserve the captured biological material according to its localization.

148. The formulation of any one of claims 108-147, wherein the formulation acts as a carrier formulation.

149. The formulation of claim 148, wherein the carrier formulation comprises encapsulated nano-particles.

150. The formulation of claim 149, wherein the encapsulated nano-particles are encapsulated in a coating that breaks down upon exposure to defined conditions for a defined time period.

151. The formulation of claim 150, wherein the defined conditions are unique to the nasal cavity, olfactory region, or targeted sub-region of the olfactory region.

152. The formulation of claim 150 or 151, wherein the defined conditions comprise temperature, pH, and/or contact with a specific biological material.

153. The formulation of any one of claims 150-152, wherein the breakdown of the coating releases a chemical configured to change the carrier-formulation from a semi-solid state to a liquid state.

154. The formulation of any one of claims 108-153, wherein the formulation comprises one or more specific mono or polyclonal antibodies so as to target a specific biological material.

155. The formulation of any one of claims 108-154, wherein the formulation comprises one or more specific aptamers so as to target a specific biological material.

156. The formulation of claim 154 or 155, wherein the specific biological material is cystatin-C.

157. The formulation of claim 154 or 155, wherein the specific biological material is a virus or a portion or derivative thereof.

158. The formulation of 157, wherein the virus is SARS CoV-2.

159. The formulation of any one of claims 108-158, comprising anti-microbial agents so as to preserve the captured biological material.

160. The formulation of claim 159, wherein the anti-microbial agents comprise 25% v/v ethanol and/or 5% w/v citric acid.

161. The formulation of any one of claims 108-160, wherein the formulation comprises microbial enriching and preservation material.

162. The formulation of claim 161, wherein the microbial enriching and preservation material comprises 25% v/v tryptic soy broth.

163. The formulation of any one of claims 108-162, wherein the formulation comprises hydrogels.

164. The formulation of any one of claims 108-163, wherein the formulation comprises sugar.

165. The formulation of any one of claims 108-164, wherein the formulation is shear thinning or shear thickening.

166. The formulation of any one of claims 108-165, wherein the formulation is immiscible with water.

167. The formulation of any one of claims 108-166, wherein the formulation is miscible with water.

168. The formulation of any one of claims 108-167, wherein the formulation is configured to preserve the biological material.

169. The formulation of any one of claims 108-168, wherein the formulation is configured to preserve an integrity of the biological material.

170. The formulation of any one of claims 108-169, wherein the formulation is configured to change into a cohesive body after delivery to the nasal cavity, the olfactory region, or the targeted sub-region of the olfactory region.

171. The formulation of claim 170, wherein the formulation comprises a solvent that evaporates to change the formulation into a cohesive body.

172. The formulation of claim 171, comprising a chemical agent that a) reacts after a time delay, b) reacts with air, c) reacts with a separately introduced gas or liquid, or d) reacts with a patient's body fluid, so as to form a cohesive body.

173. The formulation of any of one of claims 108-172, wherein the formulation is configured to absorb the biological material from the olfactory region.

174. The formulation of any one of claims 108-173, wherein the formulation is provided, delivered, and/or withdrawn as a bolus of the formulation.
